(19) United States
(12) Patent Application Publication
(10) Pub. No.: US 2024/0231392 A1
(43) Pub. Date: Jul. 11, 2024

(54) OPERATION METHOD, DEVICE, SYSTEM, AND MOBILE DEVICE FOR MEDICAL DEVICE

(71) Applicant: SHANGHAI UNITED IMAGING HEALTHCARE CO., LTD., Shanghai (CN)

(72) Inventors: CHENG NI, Shanghai (CN); WEI ZHANG, Shanghai (CN); ZHI-DU ZHANG, Shanghai (CN); XU-KUN WANG, Shanghai (CN); LI WANG, Shanghai (CN); YI-FENG WANG, Shanghai (CN)

(21) Appl. No.: 18/287,191

(22) PCT Filed: May 26, 2021
(86) PCT No.: PCT/CN2021/096085

§ 371 Date: Oct. 17, 2023

(30) Foreign Application Priority Data
Apr. 17, 2021 (WO) PCT/CN2021/087945

(51) Int. Cl. G05D 1/689; A61B 5/00; A61B 34/10; A61B 34/20; A61B 34/30; A61B 90/00; G06T 7/00; G06T 7/73; G06V 10/80; G06V 20/58; G06V 40/10

(52) U.S. Cl. CPC G05D 1/689; A61B 5/0077; A61B 5/6887; A61B 34/30; G06T 7/0012; G06T 7/73; G06V 10/806; G06V 20/58; G06V 40/10; A61B 2034/107; A61B 2034/2055; A61B 2090/366; G06T 2207/30004; G06T 2207/30252; G06V 2201/03

(57) ABSTRACT

An operation method, a device, a system and a mobile device for a medical device are disclosed. The method comprises: when it is detected that a target object reaches an operation area, projecting a first image in the operation area (S201), the first image being used for guiding the target object into a preset area; when the target object enters the preset area, acquiring position information of the target object on the preset area, and controlling, according to the position information, a target operation device to perform a first target operation (S202); and obtaining feature information of the target object in real time when the target operation device performs the first target operation, and controlling, according to the feature information, the target operation device to adjust the first target operation (S203). The accuracy of the first target operation performed by the target operation device is ensured.

### CROSS-REFERENCES TO RELATED APPLICATIONS

This application is the national phase under 35 U.S.C. 371 of PCT international application No. PCT/CN2021/096085, which has an international filing date of May 26, 2021 and claims priority of PCT international application No. PCT/CN2021/081945 filed on Apr. 17, 2021. The contents of the above identified PCT international applications are hereby incorporated in their entireties by reference for all purposes.

### TECHNICAL FIELD

The present disclosure relates to the technical field of medical devices, and more particularly, to an operation method, a device, a system, and a mobile device for a medical device.

### BACKGROUND

Currently, in the fields of medical imaging scanning and radiotherapy, the accuracy of a position of a subject before imaging or radiotherapy, and a feedback of motion monitoring during an imaging or radiotherapy process will affect the imaging quality of the subject or the efficacy of the radiotherapy. Therefore, how to provide an operation method to enable medical devices to operate and adjust in real time based on the position of the subject, and the feedback of the motion monitoring during the imaging or radiotherapy process has become an urgent technical problem that needs to be solved by persons skilled in the art.

### SUMMARY

Based on this, the present disclosure discloses an operation method, a device, a system, and a mobile device for a medical device, so as to realize that the medical device can perform a corresponding operation in real time according to the position of the subject, and the operation of the medical device is adjusted in real time according to the position of the subject and the feedback of the motion monitoring during the operation when the medical device performs the corresponding operation.

In a first aspect, an operation method is provided. The operation method includes:
projecting, when it is detected that a target object reaches an operation area, a first image in the operation area, the first image being configured to guide the target object into a preset area;
acquiring, in a case that the target object enters the preset area, position information of the target object in the preset area, and controlling, based on the position information, a target operation device to perform a first target operation; and
acquiring, during execution of the first target operation by the target operation device, feature information of the target object in real time, and controlling, based on the feature information, the target operation device to adjust the first target operation.

Preferably, the acquiring the position information of the target object in the preset area includes:
acquiring local position information of the target object in the preset area at each of at least one preset angle; and
fusing the local position information at each preset angle to obtain the position information.

Preferably, the controlling, based on the position information, the target operation device to perform the first target operation includes:
controlling, based on the position information, the target operation device to emit a target signal to an area of interest of the target object, or to adjust its own position.

Preferably, the controlling, based on the position information, the target operation device to perform the first target operation on the target object includes:
sending the position information to the target host, such that the target host controls, based on the position information, the target operation device to emit a target signal to an area of interest of the target object, or to adjust its own position.

Preferably, the controlling, based on the feature information, the target operation device to adjust the first target operation includes:
determining whether there is abnormal information in the feature information; and
controlling, if the abnormal information exists in the feature information, the target operation device to perform an adjustment operation, the adjustment operation including at least one of stopping emitting a target signal to an area of interest of the target object, stopping adjusting its own position, or reducing a frequency of the target signal emitted to the area of interest of the target object.

Preferably, the controlling, based on the feature information, the target operation device to adjust the first target operation includes:
sending the feature information to a target host, such that the target host controls the target operation device to perform an adjustment operation based on the feature information, the adjustment operation including at least one of stopping emitting a target signal to an area of interest of the target object, stopping adjusting its own position, or reducing a frequency of the target signal emitted to the area of interest of the target object.

Preferably, the acquiring the feature information of the target object in real time includes:
acquiring a feature image of the target object in real time, the feature image including at least one of an RGB image of the target object, a depth image of the target object, or an infrared image of the target object; and
obtaining, based on the feature image, the feature information.

Preferably, the method further includes:
guiding, during the execution of the first target operation by the target operation device, the target object to adjust its own breathing through a preset guidance method, the preset guidance method including displaying a second image and/or broadcasting a voice. Preferably, the method further includes:
acquiring, during the execution of the first target operation by the target operation device, a breathing signal of the target object in real time; and
sending the breathing signal to the target host, such that the target host performs a second target operation based on the breathing signal; the second target operation including at least one of imaging the target object, adjusting an emission dose of the target signal, or adjusting a surgical instrument.

Preferably, the method further includes:
guiding, when it is detected that the target object enters an operation room, the target object to move to the operation area through a preset indication method.

Preferably, the guiding the target object to move to the operation area through the preset indication method includes:
broadcasting a first voice prompt to guide the target object to move to the operation area.

Preferably, the guiding the target object to move to the operation area through the preset indication method includes: projecting a first indication sign to ground to guide the target object to move to the operation area.

Preferably, before the guiding the target object to move to the operation area through the preset indication method, the method further includes:
acquiring identity information of the target object;
determining whether the identity information matches target identity information, the target identity information being identity information acquired from the target host; and
guiding, if the identity information matches the target identity information, the target object to move to the operation area through the preset indication method.

Preferably, the method further includes:
broadcasting the identity information of the target object through voice.

Preferably, the method further includes:
broadcasting, in a case that it is detected that the target operation device has completed the first target operation, a second voice prompt, the second voice prompt being configured to prompt the target object that the first target operation has been completed.

Preferably, the method further includes:
projecting a second indication sign to the ground, the second indication sign being configured to guide the target object to leave the operation room.

Preferably, the method further includes:
acquiring, during the execution of the first target operation by the target operation device, position information of a moving part of the target object;
determining, based on the position information of the moving part, whether the target object collides with the target operation device; and
controlling, if so, the target operation device to stop executing the first target operation.

Preferably, the method further includes:
acquiring spatial environment information acquired by an acquisition device;
determining, based on the spatial environment information, whether there is an obstacle on a traveling path; and
executing, if so, an avoidance operation; the avoidance operation including at least one of the following operations: stopping moving, or replanning the traveling path based on a position of the obstacle and a destination address.

In a second aspect, an operation method for a medical device is provided. The operation method includes:
receiving position information of a target object sent by a mobile device, the position information being information acquired by the mobile device in a case that the target object enters a preset area; and
controlling, based on the position information, a target operation device to perform a first target operation.

Preferably, the controlling, based on the position information, the target operation device to perform the first target operation includes:
controlling, based on the position information, the target operation device to emit a target signal to an area of interest of the target object, or to adjust its own position.

Preferably, the method further includes:
receiving a breathing signal of the target object sent by the mobile device, the breathing signal being acquired in real time by the mobile device during execution of the first target operation by the target operation device; and
executing, based on the breathing signal, a second target operation; the second target operation including at least one of imaging the target object, adjusting an emission dose of the target signal, or adjusting a surgical instrument.

Preferably, the method further includes:
receiving feature information of the target object sent by the mobile device, the feature information being information obtained based on a feature image of the target object acquired in real time by the mobile device during execution of the first target operation by the target operation device;
determining whether there is abnormal information in the feature information; and
controlling, if the abnormal information exists in the feature information, the target operation device to perform an adjustment operation, the adjustment operation including at least one of stopping emitting a target signal to an area of interest of the target object, stopping adjusting its own position, or reducing a frequency of the target signal emitted to the area of interest of the target object.

In a third aspect, an operation system for a medical device is provided. The operation system includes a target operation device, a mobile device, and a target host. The target operation device is configured to perform a first target operation on an area of interest of a target object, and the first target operation includes imaging and/or treatment. The mobile device is movable relative to the target operation device, and configured to present and/or acquire information related to the target object. The target host is configured to control the target operation device to perform the first target operation.

Preferably, the mobile device includes a projection device configured to project a first image for guiding the target object to position.

Preferably, the mobile device further includes an acquisition device configured to acquire position information of the target object in a first orientation and a second orientation, respectively; and
the target host is configured to adjust a relative position between the target object and the target operation device, and/or control the target operation device to perform the first target operation, based on the position information of the first orientation and the position information of the second orientation.

Preferably, the mobile device includes a first mobile device and a second mobile device, the first mobile device acquires position information of the target object in a first orientation, and the second mobile device acquires position information of the target object in a second orientation.

Preferably, the projection device is further configured to project a second image for guiding the target object to adjust its own breathing.

Preferably, the acquisition device is further configured to acquire a feature image of the target object in real time, acquire, based on the feature image, feature information, and controls, based on the feature information, the target operation device to adjust the first target operation.

Preferably, the mobile device is configured to provide a preset indication manner to guide the target object to move to a preset operation area and/or to leave the operation room.

Preferably, the acquisition device is further configured to acquire a breathing signal of the target object in real time, and send the breathing signal to the target host, such that the target host performs a second target operation based on the breathing signal. The second target operation includes at least one of imaging the target object, adjusting an emission dose of the target signal, or adjusting a surgical instrument.

Preferably, the mobile device is further configured to: determine identity information of the target object; determine whether the identity information matches target identity information; and guide, if the identity information matches the target identity information, the target object to move to the operation area through the preset indication method. The target identity information is identity information acquired from the target host.

Preferably, the mobile device is further configured to: acquire position information of a moving part of the target object; determine, based on the position information of the moving part, whether the target object collides with the target operation device; and control, if so, the target operation device to stop executing the first target operation.

Preferably, the mobile device is further configured to: acquire spatial environment information acquired by an acquisition device; determine, based on the spatial environment information, whether there is an obstacle on a traveling path; and execute, if so, an avoidance operation. The avoidance operation includes at least one of the following operations: stopping moving, or replanning the traveling path based on a position of the obstacle and a destination address.

Preferably, the mobile device includes at least one of a wall crawling robot, a ground walking robot, a movable camera on a ceiling, and an unmanned aerial vehicle.

Preferably, the acquisition device includes at least one of an RGB camera, a depth camera, an infrared camera, a voice device and a light emitting device.

Preferably, the mobile device further includes a main body and a driving device for driving the main body to move.

Preferably, the mobile device further includes a trajectory generator; and
the trajectory generator is configured to generate a movement trajectory for the mobile device.

In a fourth aspect, an operation system for a medical device is provided. The operation system includes a mobile device, a target host, and a target operation device.

The mobile device is configured to perform the operation method for a medical device as described in the first aspect.

The target host is configured to perform the operation method for a medical device as described in the second aspect.

The target operation device is configured to perform a first target operation, the first target operation including the target operation device emitting a target signal to an area of interest of the target object, and/or the target operation device adjusting its own position.

In a fifth aspect, a mobile device is provided. The mobile device is configured to perform the operation method for a medical device as described in the first aspect. The mobile device is provided with at least one of an RGB camera, a depth camera, an infrared camera, a voice device, and a light emitting device.

In a sixth aspect, an operation device for a medical device is provided. The operation device includes a projection module, a first control module, and a second control module.

The projection module is configured to project, when it is detected that a target object reaches an operation area, a first image in the operation area. The first image is configured to guide the target object into a preset area.

The first control module is configured to acquire, in a case that the target object enters the preset area, position information of the target object in the preset area, and control, based on the position information, a target operation device to perform a first target operation.

The second control module is configured to acquire, during execution of the first target operation by the target operation device, feature information of the target object in real time, and control, based on the feature information, the target operation device to adjust the first target operation.

In a seventh aspect, an operation device for a medical device is provided. The operation device includes a first receiving module and a control module.

The first receiving module is configured to receive, position information of a target object sent by a mobile device. The position information is the information acquired by the mobile device in a case that the target object enters a preset area.

The control module is configured to control, based on the position information, a target operation device to perform a first target operation.

In an eighth aspect, a target host is provided. The target host is configured to perform the operation method for a medical device as described in the second aspect.

In a ninth aspect, a computer-readable storage medium is provided, having a computer program stored thereon. The computer program, when executed by a processor, perform steps of the operation method for the medical device as described in the first aspect and the second aspect.

It can be seen from the above technical solutions that, the present disclosure discloses an operation method, a device, and a system for a medical device, and a mobile device, a target host, and a storage medium. When the mobile device detects that the target object reaches the operation area, the mobile device can guide the target object to enter the preset area accurately by projecting the first image for guiding the target object into the preset area in the operation area, so that the position information of the target object in the preset area can be acquired in the case that the target object enters the preset area. Since the mobile device acquires the three-dimensional position information of the target object in the preset area, and the three-dimensional position information contains rich information, the mobile device can accurately control the target operation device to perform the first target operation in real time based on the acquired position information, and then acquire the feature information of the target object in real time during the execution of the first target operation by the target operation device. Since the feature information of the target object is acquired in real time, the mobile device can promptly control the target operation device to adjust the first target operation based on the feature information of the target object, thereby ensuring the accuracy of the first target operation performed by the target operation device.

### BRIEF DESCRIPTION OF THE DRAWINGS

In order to describe the technical solutions of the embodiments of the present disclosure or the conventional art more clearly, the accompanying drawings required for describing the embodiments or for describing the conventional art will be briefly introduced as follows. Apparently, the accompanying drawings, in the following description, illustrate merely some embodiments of the present disclosure, for a person of ordinary skill in the art, other drawings can also be obtained according to these accompanying drawings without making any creative efforts.

FIG. 1 is a schematic diagram illustrating an application environment of an operation method for a medical device according to an embodiment.

FIG. 2 is a flow diagram illustrating an operation method for a medical device according to an embodiment.

FIG. 3 is a flow diagram illustrating an operation method for a medical device according to an embodiment.

FIG. 4 is a flow diagram illustrating an operation method for a medical device according to an embodiment.

FIG. 5 is a flow diagram illustrating an operation method for a medical device according to an embodiment.

FIG. 6 is a flow diagram illustrating an operation method for a medical device according to an embodiment.

FIG. 7 is a flow diagram illustrating an operation method for a medical device according to an embodiment.

FIG. 8 is a flow diagram illustrating an operation method for a medical device according to an embodiment.

FIG. 9 is a flow diagram illustrating an operation method for a medical device according to an embodiment.

FIG. 10 is a flow diagram illustrating an operation method for a medical device according to another embodiment.

FIG. 11 is a flow diagram illustrating an operation method for a medical device according to an embodiment.

FIG. 12 is a flow diagram illustrating an operation method for a medical device according to an embodiment.

FIG. 13 is a flow diagram illustrating an operation method for a medical device according to an embodiment.

FIG. 14 is a schematic diagram illustrating an operation system for a medical device according to an embodiment.

FIG. 15 is a schematic diagram illustrating an operation system for a medical device according to an embodiment.

FIG. 16 is a schematic diagram illustrating a non-coplanar treatment system according to an embodiment.

FIG. 17 is a schematic diagram illustrating an operation system for a medical device according to an embodiment.

FIG. 18 is a schematic diagram illustrating a mobile device according to an embodiment.

FIG. 19 is a schematic diagram illustrating a configuration of an operation device according to an embodiment.

FIG. 20 is a schematic diagram illustrating a configuration of an operation device according to an embodiment.

FIG. 21 is a schematic diagram illustrating a configuration of a target host according to an embodiment.

### DETAILED DESCRIPTION OF THE EMBODIMENTS

The technical solutions of embodiments of the present disclosure will be described clearly and completely below with reference to the accompanying drawings of the embodiments of the present disclosure. Apparently, the described embodiments are a part of but not all of the embodiments of the present disclosure. Based on the embodiments in the present disclosure, all other embodiments obtained without any creative efforts by a person of ordinary skill in the art fall within the scope of protection of the present disclosure.

The embodiment of the present disclosure discloses an operation method, a device, a system, a mobile device and a storage medium for a medical device, so as to realize that the medical device can adjust the operation of the medical device in real time based on the position of the subject and the feedback of the motion monitoring during the operation=process.

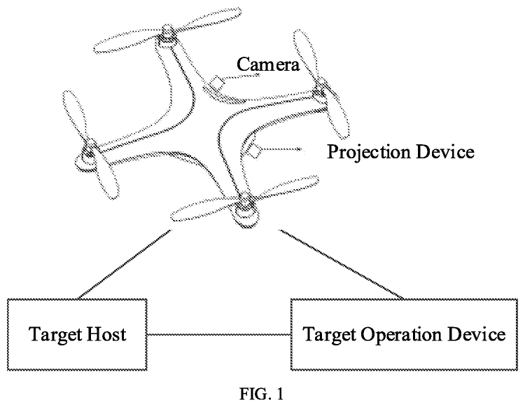

The operation method for a medical device according to the embodiment of the present disclosure may be applicable to a system shown in FIG. 1. The system includes a mobile device, a target host, and a target operation device. The mobile device communicates wirelessly with the target host and the target operation device, respectively. The mobile device may include a device having a moving capability such as an unmanned aerial vehicle, a mobile robot, a movable device, a wall slide rail robot, or the like. The target host may include a server, a personal computer, or other terminal devices. The target operation device may include an X-ray digital radiography (DR) equipment, a computed tomography (CT) equipment, a magnetic resonance imaging (MRI) equipment, a positron emission tomography (PET) equipment, a medical electron linear accelerator (LINAC) equipment, a gamma knife, a surgical robot, and so on, which is not limited thereto. It should be noted that the mobile device in FIG. 1 is only described by taking the unmanned aerial vehicle as an example and is not limited thereto.

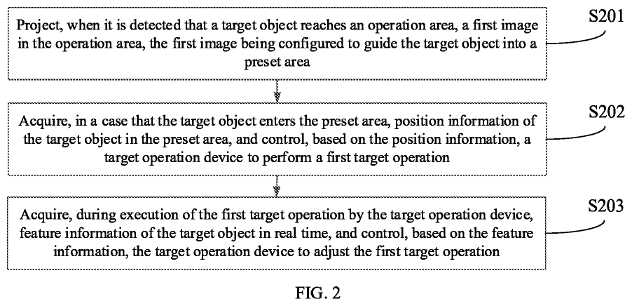

In a first aspect, in an embodiment, as shown in FIG. 2, a flow diagram illustrating an operation method for a medical device is provided. An example where the method is applied to the mobile device in FIG. 1 will be described. The method includes the following steps.

In step S201, a first image is projected in an operation area when it is detected that a target object reaches the operation area. The first image is configured to guide the target object into a preset area.

The operation area may be an operation area of a target operation device. The target operation device may include an X-ray digital radiography (DR) equipment, a computed tomography (CT) equipment, a magnetic resonance imaging (MRI) equipment, a positron emission tomography (PET) equipment, a medical electron linear accelerator, a gamma knife, a surgical robot, and so on. The preset area may include a bed area, such as a scanning bed of the target operation device or a treatment bed of the target operation device, or an operation area of the surgical robot. Specifically, when detecting that the target object reaches the operation area, the mobile device projects the first image in the operation area, and guides the target object into the preset area through the first image. Optionally, the projected first image may be a virtual two-dimensional or three-dimensional image. Exemplarily, the projected first image may be a virtual three-dimensional image that guides the target object how to position himself/herself on a hospital bed. Optionally, the mobile device may acquire a depth image of the target object, and detect whether the target object has reached the above operation area through the depth image of the target object. Optionally, when detecting that the target object reaches the operation area, the mobile device may also send control instructions to other devices, to activate other devices to display images to guide the target object into the preset area. Optionally, the display image may be displayed in the above operation area, or may be displayed in a non-operation area. Optionally, the display image may be displayed on a display screen or projected by a projection device. Optionally, if there are a plurality of operation devices (such as, the DR equipment, the CT equipment, the MRI equipment, etc.) in the above operation area, information related to the operation that the target object will perform may be acquired, and then which of a plurality of indication images to be displayed can be determined, so as to guide the above target object reach the corresponding target operation device. The plurality of indication images are indication images corresponding to the above plurality of operation devices. For example, the above information may be acquired by a doctor, or scanning protocol information or information related to a treatment plan of the target object may be acquired, thereby determining a corresponding indication image for the target object.

In step S202, position information of the target object in the preset area is acquired in a case that the target object enters the preset area, and a target operation device is controlled to perform a first target operation based on the position information.

Specifically, after projecting the first image in the above operation area and guiding the target object into the preset area, the mobile device acquires the position information of the target object in the above preset area, and controls the target operation device to perform the first target operation based on the acquired position information. Optionally, the first target operation in this embodiment may be to emit a target signal to an area of interest of the above target object, for example, an X-ray signal may be emitted to the area of interest of the above target object for CT or X-ray imaging, or a pulse sequence may be emitted to the area of interest of the above target object for magnetic resonance imaging, or radiation may be emitted to the area of interest of the above target object to perform radiation therapy on the target object. The radiation may include electrons, photons, protons or heavy ions. Alternatively, a surgical instrument may be operated to operate on the area of interest of the above target object. Optionally, the first target operation in this embodiment may also include that the target operation device adjusts its own position. Optionally, the mobile device may control the target operation device to perform the above first target operation when the position information of the target object on the above preset area meets operation requirements. Exemplarily, taking the first target operation being to emit the target signal to the area of interest of the target object as an example, the mobile device may control the target operation device to emit the target signal to the area of interest of the target object in a case that the area of interest of the target object coincides with the emission position where the target operation device emits the target signal.

In step S203, feature information of the target object is acquired in real time during execution of the first target operation by the target operation device, and the target operation device is controlled to adjust the first target operation based on the feature information.

Specifically, during the execution of the first target operation by the target operation device, the mobile device acquires the feature information of the target object in real time, and controls the target operation device to adjust the first target operation based on the feature information of the target object. Optionally, the feature information of the target object in this embodiment may be the position information of the target object, facial expression information of the target object, or a breathing signal of the target object. Optionally, the mobile device may acquire a feature image such as a facial image of the target object during the execution of the first target operation by the target operation device, and obtain the feature information of the target object from the acquired feature image of the target object, and the mobile device may also send an acquisition instruction to the target operation device to acquire the feature information of the target object from the target operation device. Optionally, the mobile device may analyze the acquired feature information of the target object, and control the above target operation device to adjust the above first target operation when it is determined that there is abnormal information in the feature information of the target object. Optionally, the mobile device may control the target operation device to adjust the first target operation by controlling the target operation device to suspend the execution of the first target operation. It should also be noted that if the target operation device is a radiotherapy equipment, the application of radiotherapy includes a non-coplanar treatment. In this scenario, the treatment bed of the target operation device is at an unconventional angle, for example, the treatment bed may be rotated to different angles around a vertical axis. The mobile device may track the angle of the treatment bed and acquire a current position and posture of the target object in real time at a current angle of the treatment bed, and calculate a difference between the current position and posture of the target object and a planned position and posture, and compare the difference with a preset threshold. The threshold may be adjusted by a doctor, a physicist or a technician according to a condition of the target object and different imaging or treatment sites. If a real-time position deviation of the patient calculated by the mobile device exceeds the preset threshold, the target operation device will be controlled to terminate the treatment process, and the treatment process will be restarted after the technician redetermines the position accurately by moving a six-dimensional bed.

Further, in addition to acquiring the position information of the target object for determining the position and acquiring the feature information of the target object for monitoring the first target operation performed by the target operation device, the mobile device may also use the acquired feature information of the target object to perform anti-collision monitoring on a moving part of the target operation device during the execution of the first target operation by the target operation device. The mobile device may also prompt the target object through a voice broadcast based on an anti-collision monitoring result to avoid collision between the target object and the target operation device. Exemplarily, assuming that the feature information of the target object is the position information of the target object and the first target operation performed is a radiotherapy operation, the mobile device may monitor the collision between a treatment head of the medical electron linear accelerator and the target object during radiotherapy, or monitor the collision between the treatment head of the medical electron linear accelerator and the treatment bed, or monitor the collision between an electronic portal imaging device (EPID) of the medical electron linear accelerator and the target object, etc.

In this embodiment, when the mobile device detects that the target object reaches the operation area, the mobile device can guide the target object to enter the preset area accurately by projecting the first image for guiding the target object into the preset area in the operation area, so that the position information of the target object in the preset area can be acquired. Since the mobile device acquires the three-dimensional position information of the target object in the preset area, and the three-dimensional position information contains rich information, the mobile device can accurately control the target operation device to perform the first target operation in real time based on the acquired position information, and then acquire the feature information of the target object in real time during the execution of the first target operation by the target operation device. Since the feature information of the target object is acquired in real time, the mobile device can promptly control the target operation device to adjust the first target operation based on the feature information of the target object, thereby ensuring the accuracy of the first target operation performed by the target operation device.

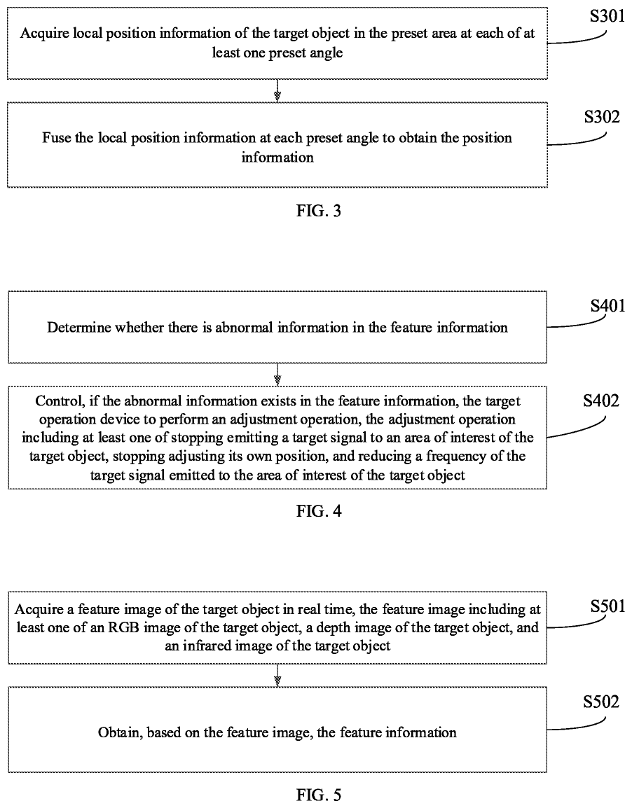

In the above scenario where the mobile device acquires the position information of the target object in the preset area, the mobile device may acquire the position information of the target object in the preset area from a plurality of angles. In an embodiment, as shown in FIG. 3, a flow diagram illustrating another operation method is provided. Based on the above embodiment, as an optional implementation, the acquiring the position information of the target object in the preset area in the above step S202 includes the following steps.

In step S301, local position information of the target object in the preset area is acquired at each of at least one preset angle.

Specifically, the mobile device acquires the local position information of the target object in the preset area at each of at least one preset angle. Exemplarily, the above mobile device may acquire local positions of the above target object in the preset area on a left side, a right side, and a front side of the above target operation device, respectively. It should be noted here that, when using the mobile device to acquire the local position information of the target object in the preset area at different angles, the same mobile device may be used to acquire the local position of the target object in the preset area at the corresponding angle from different angles, or two or more mobile devices may be used to simultaneously acquire the local positions of the target object in the preset area from different angles. For example, when the mobile device moves from one angle to another angle, there will be a time difference, so that two or three mobile devices may be used to simultaneously acquire the local position information of the target object in the above preset area at different angles, thereby improving the accuracy of the position.

In step S302, the local position information at each preset angle is fused to obtain the position information.

Specifically, the mobile device fuses the above local position information at each preset angle to obtain the position information of the target object in the above preset area. Optionally, the mobile device may fuse the local position information at each preset angle by using a fusion algorithm to obtain the position information of the target object in the above preset area, or fuse the local position information acquired at each preset angle by using a splicing method to obtain the position information of the target object in the above preset area. Optionally, the mobile device may also send the local position information at each preset angle to a processor capable of communicating with the mobile device, and the processor fuses all the local position information to obtain the position information of the target object in the above preset area. Then the processor sends the position information of the target object in the above preset area to the mobile device, and the mobile device receives the position information.

In this embodiment, since the local position information acquired by the mobile device is the position information of the target object in the preset area acquired from a plurality of preset angles, and the local position information acquired from different angles is different, the obtained position information includes the position information at different angles after fusing the local position information at all of the preset angles, which makes the obtained position information relatively complete and ensures the accuracy of the obtained position information of the target object in the preset area.

In the scenario where the above mobile device controls the target operation device to perform the first target operation based on the position information of the target object in the preset area, the mobile device may control the target operation device to perform the above first target operation based on the position information of the target object in the preset area. The mobile device may also send the position information of the target object in the preset area to the target host, and then the target host controls the target operation device to perform the above first target operation based on the position information of the target object in the preset area. The embodiments corresponding to these two scenarios are described separately below.

In a first scenario, the mobile device controls the target operation device to perform the above first target operation based on the position information of the target object in the preset area. In an embodiment, on the basis of the above embodiment, as an optional implementation, the controlling the target operation device to perform the first target operation based on the position information of the target object in the preset area in the above step S202 includes: controlling, based on the position information, the target operation device to emit a target signal to an area of interest of the target object, or to adjust its own position.

Specifically, the mobile device controls the target operation device to emit the target signal to the area of interest of the target object, or controls the target operation device to adjust its own position, based on the position information of the target object in the preset area. Optionally, the mobile device may determine a matching result between the position information of the target object in the preset area and preset reference position information. Optionally, the mobile device may acquire the preset reference position information corresponding to the above target operation device in advance, and store the preset reference position information in its own memory. After acquiring the position information of the target object in the above preset area, the matching result between the acquired position information and the preset reference position information is determined. Optionally, the mobile device may compare the above acquired position information with the above preset reference position information to determine the matching result between the acquired position information and the above preset reference position information. The mobile device may also register the acquired position information and the above preset reference position information to determine the matching result between the acquired position information and the above preset reference position information. Optionally, the matching result obtained by the mobile device may include that the above acquired position information matches the preset reference position information, the above acquired position information does not match the preset reference position information, or a deviation between the above acquired position information and the preset reference position information. Further, based on the matching result obtained above, the mobile device controls the target operation device to emit the target signal to the area of interest of the target object, or controls the target operation device to adjust its own position, i.e., the above first target operation may include emitting the target signal to the area of interest of the target object (for example, performing imaging or radiotherapy on the area of interest of the target object), or controlling the target operation device to adjust its position. That is to say, if the matching result obtained by the mobile device is that the above acquired position information matches the preset reference position information, the mobile device controls the above target operation device to emit the target signal to the area of interest of the target object. If the matching result obtained by the mobile device is that the above acquired position information does not match the preset reference position information, the mobile device controls the target operation device to adjust its own position, for example, a position of the bedplate of the hospital bed is adjusted based on the above deviation, so that the acquired position information matches the preset reference position information. Optionally, the area of interest of the target object may be the abdomen of the target object, or the chest of the target object, or the like. Optionally, the area of interest may include tumors. Optionally, the target signal emitted by the target operation device to the area of interest of the target object may include an X-ray signal, a pulse sequence, or radiation. It should be understood that the target signals emitted by different types of target operation devices are also different. If the target operation device is the DR equipment, the target signal emitted by the target operation device is the X-ray signal. If the target operation device is the MRI equipment, the target signal emitted by the target operation device is an MRI signal. If the target operation device is the radiotherapy equipment, the target signal emitted by the target operation device is the radiation. It should be noted that, if the target operation device is the radiotherapy equipment, and the application of radiotherapy includes a non-coplanar treatment. In this scenario, the mobile device may track the angle of the treatment bed and acquire a position and posture of the target object in real time at a current angle of the treatment bed, and calculate a difference between the current position and posture of the target object and a planned position and posture, and compare the difference with a preset threshold. The threshold may be adjusted by a doctor, a physicist or a technician according to a condition of the target object and different imaging or treatment sites. If a real-time position deviation of the patient calculated by the mobile device exceeds the preset threshold, the target operation device will be controlled to terminate the treatment process, and the treatment process will be restarted after the technician redetermines the position accurately by moving a six-dimensional bed.

In this embodiment, after acquiring the position information of the target object in the preset area, the mobile device controls, based on the position information, the target operation device to emit the target signal to the area of interest of the target object, or to adjust its own position. Since the mobile device controls the target operation device to perform the operation based on the position information of the target object in the preset area, the controlling the target operation device to perform the corresponding operation based on the position information of the target object in the preset area can ensure the accuracy of the operation performed by the target operation device controlled by the mobile device.

In a second scenario, the mobile device sends the position information of the target object in the preset area to the target host, and the target host controls the target operation device to perform the above first target operation based on the position information of the target object in the preset area. In an embodiment, on the basis of the above embodiment, as an optional implementation, the controlling the target operation device to perform the first target operation based on the position information of the target object in the preset area in the above S202 includes: sending the position information to the target host, such that the target host controls, based on the position information, the target operation device to emit a target signal to an area of interest of the target object, or to adjust its own position.

Specifically, after acquiring the position information of the target object in the preset area, the mobile device sends the acquired position information of the target object in the preset area to the target host, so that the target host controls the target operation device to emit the target signal to the area of interest of the target object, or control the target operation device to adjust its own position, based on the position information sent by the mobile device and the preset reference position information. It should be noted that, the process of the mobile device sending the acquired position information of the target object in the preset area to the target host is a real-time sending process. Similarly, if the target host determines that the position information acquired by the above mobile device matches the preset reference position information, the target host controls the above target operation device to emit the target signal to the area of interest of the target object. If the target host determines that the position information acquired by the above mobile device does not match the preset reference position information, the target host controls the target operation device to adjust its own position. Optionally, the area of interest of the target object may be the abdomen of the target object, or the chest of the target object, or the like. Optionally, the target signal emitted by the target operation device to the area of interest of the target object may include an X-ray signal, a magnetic resonance signal, or radiation. It should be understood that the target signals emitted by different types of target operation devices are also different. If the target operation device is the DR equipment, the target signal emitted by the target operation device is the X-ray signal. If the target operation device is the MRI equipment, the target signal emitted by the target operation device is an MRI signal. If the target operation device is the radiotherapy equipment, the target signal emitted by the target operation device is the radiation. It should be noted that, if the target operation device is the radiotherapy equipment, and the application of radiotherapy includes a non-coplanar treatment, in this scenario, the treatment bed of the target operation device is at an unconventional angle, for example, the treatment bed can be rotated to different angles around a vertical axis. The mobile device may track the angle of the treatment bed and acquire a position and posture of the target object in real time at a current angle of the treatment bed, and send the acquired position and posture of the target object to the target host. Then the target host calculates a difference between the current position and posture of the target object and a planned position and posture, and compare the difference with a preset threshold. The threshold may be adjusted by a doctor, a physicist or a technician according to a condition of the target object and different imaging or treatment sites. If a real-time position deviation of the patient calculated by the target host exceeds the preset threshold, the target operation device will be controlled to terminate the treatment process, and the treatment process will be restarted after the technician redetermines the position accurately by moving a six-dimensional bed.

In this embodiment, after acquiring the position information of the target object in the preset area, the mobile device sends the position information of the target object in the preset area to the target host, so that the target host may control the target operation device to transmit the target signal to the area of interest of the target object, or control the target operation device to adjust its own position, based on the acquired position information and the preset reference position information. Since the target host controls the target operation device to perform the operation based on the position information of the target object in the preset area acquired by the mobile device, the controlling the target operation device to perform the corresponding operation based on the position information of the target object in the preset area can ensure the accuracy of the operation performed by the target operation device controlled by the target host.

In the scenario where the above mobile device controls the target operation device to adjust the first target operation based on the feature information of the target object, the mobile device may control the target operation device to adjust the above first target operation based on the feature information of the target object, or the mobile device may send the feature information of the target object to the target host, and the target host controls the target operation device to adjust the above first target operation based on the feature information of the target object. The embodiments corresponding to these two scenarios are described separately below.

In a third scenario, the mobile device controls the target operation device to adjust the above first target operation based on the feature information of the target object. In an embodiment, as shown in FIG. 4, a flow diagram illustrating another operation method is provided. On the basis of the above embodiment, as an optional implementation, the controlling the target operation device to adjust the first target operation based on the feature information of the target object in the above step S203 includes the following steps.

In step S401, whether there is abnormal information in the feature information is determined.

Specifically, the mobile device determines whether there is abnormal information in the acquired feature information of the target object. Optionally, the feature information of the target object may include the position information of the target object, the facial expression of the target object, the breathing signal of the target object, and so on. Exemplarily, the abnormal information in the feature information of the target object may include the position information of the target object deviating from preset position information, the facial expression of the target object being an abnormal facial expression, and the breathing signal of the target object being relatively rapid. Optionally, the mobile device may determine whether there is the abnormal information in the feature information of the target object through a recognition network, or determine whether there is the abnormal information in the feature information of the target object by classifying the feature information of the target object through a classifier. Optionally, the recognition network may be a machine learning model or a deep learning model.

In step S402, the target operation device is controlled to perform an adjustment operation if the abnormal information exists in the feature information. The adjustment operation includes at least one of stopping emitting the target signal to the area of interest of the target object, stopping adjusting its own position, or reducing a frequency of the target signal emitted to the area of interest of the target object.

Specifically, if it is determined that the abnormal information exists in the feature information of the target object, the mobile device controls the above target operation device to perform the adjustment operation. The adjustment operation may include at least one of stopping emitting the target signal to the area of interest of the target object, stopping adjusting its own position, or reducing the frequency of the target signal emitted to the area of interest of the target object. Exemplarily, if the mobile device determines that the position information of the target object has deviated from the preset position information, the mobile device controls the target operation device to stop adjusting its own position and stop emitting the target signal to the area of interest of the target object. If the mobile device determines that the facial expression of the target object is the abnormal facial expression, or the breathing signal of the target object is relatively rapid, the mobile device may control the target operation device to stop emitting the target signal to the area of interest of the target object, or reduce the frequency of the target signal emitted to the area of interest of the target object. Optionally, if the mobile device determines that there is the abnormal information in the above feature information, alarm information may also be provided for alarming. Optionally, the alarm information may include acoustic information, text information, optical information, etc. Optionally, after receiving the alarm information, the user can determine whether to stop the machine for inspection and adjust the target operation, or provide the alarm information simultaneously and shut down the machine. Optionally, the alarm information may be provided by the mobile device.

In this embodiment, the mobile device determines whether there is the abnormal information in the feature information of the target object, and if the abnormal information exists in the feature information of the target object, the mobile device controls the target operation device to perform at least one of stopping emitting the target signal to the area of interest of the target object, stopping adjusting its own position, or reducing the frequency of the target signal emitted to the area of interest of the target object. Since the mobile device controls the target operation device to perform the operation based on the feature information of the target object, the controlling the target operation device to perform the corresponding operation based on whether there is the abnormal information in the feature information of the target object can ensure the accuracy of the operation performed by the target operation device controlled by the mobile device.

In a fourth scenario, the mobile device sends the feature information of the target object to the target host, and the target host controls the target operation device to adjust the above first target operation based on the feature information of the target object. In an embodiment, on the basis of the above embodiment, as an optional implementation, the controlling the target operation device to adjust the first target operation based on the feature information of the target object in the above step S203 includes: sending the feature information to the target host, such that the target host controls the target operation device to perform an adjustment operation based on the feature information. The adjustment operation includes at least one of stopping emitting the target signal to the area of interest of the target object, stopping adjusting its own position, or reducing the frequency of the target signal emitted to the area of interest of the target object.

Specifically, the mobile device sends the acquired feature information of the target object to the target host, so that the target host controls the target operation device to perform the adjustment operation based on the feature information of the target object. The adjustment operation includes at least one of stopping emitting the target signal to the area of interest of the target object, stopping adjusting its own position, or reducing the frequency of the target signal emitted to the area of interest of the target object. Exemplarily, if the target host determines that the position information of the target object has deviated from the preset position information, the target host controls the target operation device to stop adjusting its own position and stop emitting the target signal to the area of interest of the target object. If the target host determines that the facial expression of the target object is the abnormal facial expression, or the breathing signal of the target object is relatively rapid, the target host may control the target operation device to stop emitting the target signal to the area of interest of the target object, or reduce the frequency of the target signal emitted to the area of interest of the target object. Optionally, if the target host determines that there is the abnormal information in the above feature information, alarm information may also be provided for alarming. Optionally, the alarm information may include acoustic information, text information, optical information, etc. Optionally, after receiving the alarm information, the user can determine whether to stop the machine for inspection and adjust the target operation, or provide the alarm information simultaneously and shut down the machine. Optionally, the alarm information may be provided by the target host.

In this embodiment, after acquiring the feature information of the target object, the mobile device sends the feature information of the target object to the target host, so that the target host may control the target operation device to perform at least one of stopping emitting the target signal to the area of interest of the target object, stopping adjusting its own position, or reducing the frequency of the target signal emitted to the area of interest of the target object based on the feature information of the target object. Since the target host controls the target operation device to perform the operation based on the feature information of the target object, the controlling the target operation device to perform the corresponding operation based on whether there is the abnormal information in the feature information of the target object can ensure the accuracy of the operation performed by the target operation device controlled by the target host.

In the scenario where the mobile device acquires the feature information of the target object in real time, the mobile device obtains the feature information of the target object by acquiring the feature image of the target object. In an embodiment, as shown in FIG. 5, a flow diagram illustrating another operation method is provided. On the basis of the above embodiment, as an optional implementation, the acquiring the feature information of the target object in real time in the above step S203 includes the following steps.

In step S501, a feature image of the target object is acquired in real time. The feature image includes at least one of an RGB image of the target object, a depth image of the target object, or an infrared image of the target object.

Specifically, the mobile device acquires the feature image of the above target object in real time, and the acquired feature image of the target object includes at least one of the RGB image of the target object, the depth image of the target object, and the infrared image of the target object. Optionally, the mobile device may acquire the RGB image of the target object through its own optical camera, acquire the depth image of the target object through its own depth camera, or acquire the infrared image of the target object through its own infrared camera. Optionally, the feature image of the target object may be a feature image of the face of the target object, or a feature image of the abdomen of the target object, etc., which is not limited thereto.

In step S502, the feature information is obtained based on the feature image.

Specifically, the mobile device obtains the feature information of the target object based on the above acquired feature image of the target object. Optionally, the mobile device may perform feature extraction on the above feature image of the target object to obtain the feature information of the target object. For example, the mobile device may determine contour information of the target object through the depth image of the target object, and then determine the breathing signal of the target object. The mobile device may also determine a change in a temperature at the mouth or nose of the target object through the infrared image to obtain the breathing signal of the target object. Alternatively, the mobile device may determine the position information of the target object through the depth image of the target object, or the mobile device may determine the facial expression information of the target object through the RGB image of the target object or the infrared image of the target object. Optionally, the mobile device may input the RGB image of the target object or the infrared image of the target object into a pre-trained recognition network, and determine the facial expression information of the target object through this recognition network.

In this embodiment, the mobile device can obtain the feature image of the target object in real time by acquiring the target object in real time, so that the mobile device can obtain the feature information of the target object based on the feature image of the target object. Since the calculation amount of this process is small and the operation is relatively simple, the mobile device can quickly obtain the feature information of the target object, and the efficiency of obtaining the feature information of the target object by the mobile device is improved.

During the above execution of the first target operation by the target operation device, a motion caused by the breathing action of the target object may affect the first target operation, so it is necessary to guide the target object to adjust his/her own breathing action. In an embodiment, on the basis of the above embodiment, as an optional implementation, the above method further includes: guiding, during the execution of the first target operation by the target operation device, the target object to adjust his/her own breathing through a preset guidance method. The preset guidance method includes displaying a second image and/or broadcasting a voice.

Specifically, the mobile device guides the target object to adjust his/her own breathing through the preset guidance method during the execution of the above first target operation by the above target operation device. The preset guidance method includes displaying the second image and/or the voice broadcast. Optionally, the second image may be displayed on the top of a preset position corresponding to the preset position information or on a ceiling perpendicular to a viewing angle of the eyes of the target object, and the target object may be guided to adjust his/her own breathing through the second image. For example, the target object is guided to perform a deep inspiration breath-hold technique (DIBH for short) by projecting the second image, and then perform imaging or treatment during the breath-hold period. It should be noted that, the mobile device guiding the target object to adjust his/her own breathing through the second image or the voice broadcast may include guiding the target object to adjust his/her own breathing frequency, or guiding the target object to adjusting his/her own breathing action. Optionally, the second image may include a breathing frequency signal, or a virtual two-dimensional or three-dimensional breathing image. Optionally, as an implementation, when the mobile device guides the target object to adjust his/her own breathing action by projecting the second image on the top of the preset position, the mobile device may further guide the target object to adjust his/her own breathing action by broadcasting a corresponding guidance sound. Optionally, as another optional implementation, the mobile device may also guide the target object to adjust his/her own breathing only by displaying the second image, or the mobile device may also guide the target object to adjust his/her own breathing only by the voice broadcast.

In this embodiment, the mobile device can guide the target object to adjust his/her own breathing through the preset guidance method during the execution of the first target operation by the target operation device, reducing an impact of breathing of the target object on the execution of the first target operation.

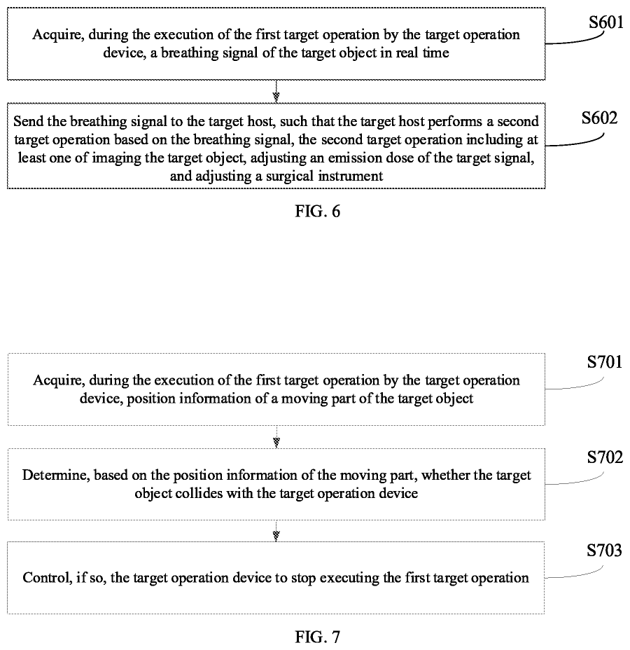

During the above execution of the first target operation by the target operation device, the mobile device may also acquire the breathing signal of the target object in real time, and send the acquired breathing signal of the target object to the target host, so that the target host may perform a corresponding operation based on the breathing signal of the target object during the above execution of the first target operation. In an embodiment, as shown in FIG. 6, a flow diagram illustrating another operation method is provided. On the basis of the above embodiment, as an optional implementation, the above method further includes the following steps.

In step S601, a breathing signal of the target object is acquired in real time during the execution of the first target operation by the target operation device.

Specifically, the mobile device acquires the breathing signal of the target object in real time during the above execution of the first target operation by the target operation device. Optionally, the mobile device may acquire the depth image of the target object and the infrared image of the target object through its own depth camera and infrared camera, and obtain the breathing signal of the target object through the depth image of the target object and the infrared image of the target object. For example, the mobile device may determine contour information of the target object through the depth image of the target object, and then determine the breathing signal of the target object. The mobile device may also determine a change in a temperature at the mouth and nose of the target object through the infrared image to obtain the breathing signal of the target object.

In step S602, the breathing signal is sent to the target host, such that the target host performs a second target operation based on the breathing signal. The second target operation includes at least one of imaging the target object, adjusting an emission dose of the target signal, or adjusting a surgical instrument.

Specifically, the mobile device sends the breathing signal of the target object to the target host, such that the target host performs the second target operation based on the breathing signal of the target object. The second target operation performed by the target host includes at least one of imaging the target object, adjusting the emission dose of the target signal, or adjusting the surgical instrument. It should be understood that, if the second target operation performed by the target host based on the breathing signal of the target object is to image the target object based on the breathing signal of the target object, the corresponding first target operation may include performing at least one of an X-ray scan, a CT scan, an MR scan, a PET scan, or an ultrasound scan on the target object. Optionally, in this embodiment, if the second target operation performed by the target host is to image the target object based on the breathing signal of the target object, the imaging method may be a prospective imaging method or a retrospective imaging method. For example, in CT imaging, the imaging scan may be performed at a specific respiratory phase based on the breathing signal, or the imaging scan data may be acquired at a plurality of respiratory cycles, and then a corresponding image may be reconstructed by extracting the imaging scan data at different respiratory phases, which can effectively reduce motion artifacts and improve the quality of imaging. Optionally, in this embodiment, the target host may also control the emission of the target signal based on the breathing signal of the target object. For example, in the radiotherapy, when the mobile device monitors that the target object is at the specific respiratory phase based on the breathing signal, the output of the radiotherapy device can be controlled, thereby improving an efficiency of the treatment and reducing radiation damage to normal organs and tissues. Optionally, in the radiotherapy, the target operation device may perform the first and second target operations on the target object based on a four-dimensional (4D) radiotherapy plan. In this case, the target host may also monitor and control the output of a treatment beam of the radiotherapy device based on the breathing signal of the target object acquired in real time by the mobile device and the breathing signal in the four-dimensional radiotherapy plan in the radiotherapy device. If a deviation between the real-time acquired signal and the signal in the four-dimensional radiotherapy plan exceeds a threshold, the radiotherapy device is controlled to stop the treatment.

In this embodiment, the mobile device acquires the breathing signal of the target object in real time during the execution of the first target operation by the target operation device, so that the target host can image the target object based on this breathing signal or adjust the emission dose of the target signal based on this breathing signal. As a result, the first target operation performed by the target operation device can be flexibly adjusted or the target object can be more accurately imaged based on the breathing signal of the target object.

In the above scenario where the mobile device detects that the target object reaches the operation area, the target object needs to be firstly guided to move to the operation area. On the basis of the above embodiment, in an embodiment, the above method further includes: guiding, when it is detected that the target object enters an operation room, the target object to move to the operation area through a preset indication method.

Specifically, when detecting that the target object enters the operation room, the mobile device guides the target object to move to the above operation area through the preset indication method. Optionally, the mobile device may guide the target object to move to the operation area by broadcasting a first voice prompt, or may guide the target object to move to the operation area by projecting a first indication sign to ground. Optionally, the mobile device may acquire an image of a wristband on the hand of the target object. If the image of the wristband of the target object is acquired, it is determined that the target object has entered the operation room. Optionally, the target object may carry a positioning device that can send position information, and the mobile device may acquire this position information to determine whether the target object enters the operation room. Optionally, the mobile device may also determine whether the target object enters the operation room through its own infrared sensing device. It should be understood that an indication sign for guiding the target object to move to the above operation area may also be arranged on the wall of the operation room, and the target object may be guided to move to the above operation area through this indication sign.

Further, as an optional implementation, before guiding the target object to move to the above operation area, the mobile device may verify identity information of the target object to ensure that the information of the entered target object is consistent with that of the object loaded by the technician. Specifically, the mobile device acquires the identity information of the above target object, and determines whether the identity information of the above target object matches target identity information. If the identity information of the target object matches the target identity information, the target object is guided to move to the above operation area through the above indication method. Optionally, the mobile device may also prompt the target object to verify their identity information by broadcasting the identity information of the target object through voice. Optionally, the mobile device may acquire the image of the wristband of the target object, and analyze the image of the wristband of the target object to obtain the identity information of the target object. Optionally, the mobile device may also instruct the target object to submit his/her identity information by sending a voice instruction to the target object.

In this embodiment, when detecting that the target object enters the operation room, the mobile device can quickly guide the target object to move to the operation area through the preset indication method. In addition, the target object can be guided to move to the operation area more accurately through the indication method, thereby ensuring the accuracy of the target object moving to the operation area.

When the target operation device has completed the first target operation on the target operation object, the mobile device may use a voice prompt to prompt the target object that the first target operation has been completed. On the basis of the above embodiment, in an embodiment, the above method further includes: broadcasting, in a case that it is detected that the target operation device has completed the first target operation, a second voice prompt. The second voice prompt is configured to prompt the target object that the first target operation has been completed.

Specifically, in the case that it is detected that the target operation device has completed the above first target operation, the mobile device broadcasts the second voice prompt to prompt the target object that the first target operation performed by the target operation device has been completed. Optionally, the mobile device may detect whether the target operation device has completed the first target operation by receiving a feedback signal sent by the target operation device. Optionally, the mobile device may also acquire an image of the target operation device, and determine whether the target operation device has completed the first target operation through this image.

Further, the mobile device may also project a second indication sign to ground to guide the target object to leave the above operation room. Optionally, the mobile device may also guide the target object to leave the above operation room by broadcasting a voice prompt. Optionally, as an implementable implementation, an indication sign for guiding the target object to leave the operation room may also be arranged on the wall of the operation room, and the target object may be guided to leave the operation room according to the indication sign on the wall.

In this embodiment, when the mobile device detects that the target operation device has completed the first target operation, the second voice prompt for prompting the target object that the first target operation has been completed is broadcasted, so that the target object can be promptly prompted that the first target operation has been completed.

During the above execution of the first target operation by the target operation device, the mobile device may also acquire position information of a moving part of the target object, and execute anti-collision monitoring on the moving part of the target object. In an embodiment, as shown in FIG. 7, the above method further includes the following steps.

In step S701, position information of a moving part of the target object is acquired during the execution of the first target operation by the target operation device.

Specifically, the mobile device acquires the position information of the moving part of the target object during the execution of the above first target operation by the target operation device. Optionally, the moving part of the target object may include the head of the target object, the legs of the target object, or the hands of the target object. Optionally, the mobile device may acquire an image of the moving part of the target object through its own RGB camera during the execution of the first target operation by the target operation device, and obtain the position information of the moving part of the target object based on the image of the moving part of the target object.

In step S702, whether the target object collides with the target operation device is determined based on the position information of the moving part.

Specifically, the mobile device determines whether the target object collides with the target operation device based on the position information of the moving part of the target object. Optionally, the mobile device may determine whether the target object collides with the target operation device based on a deviation between the position information of the moving part of the target object and the position information of the target operation device. Optionally, the mobile device may also determine whether the target object collides with the target operation device based on a distance between a position of the moving part of the target object and a position of the target operation device. Exemplarily, taking the target operation device being the medical electron linear accelerator and the moving part of the target object being the head of the target object as an example, the mobile device may determine whether the head of the target object collides with the medical electron linear accelerator based on the position information of the head of the target object.

In step S703, if so, the target operation device is controlled to stop executing the first target operation.

Specifically, if the mobile device determines that the target object collides with the target operation device, the mobile device controls the target operation device to stop executing the above first target operation. Optionally, the mobile device may send a control instruction to the target operation device to instruct the target operation device to stop executing the above first target operation.

In this embodiment, during the execution of the first target operation by the target operation device, the mobile device can determine whether the target object collides with the target operation device based on the position information of the moving part of the target object by acquiring the position information of the moving part of the target object, so that the target object can be timely performed anti-collision monitoring. In a case that it is determined that the target object has collided with the target operation device, the target operation device can be promptly controlled to stop executing the first target operation, thus avoiding risks to the target object and ensuring the safety of the target operation device performing the first target operation.

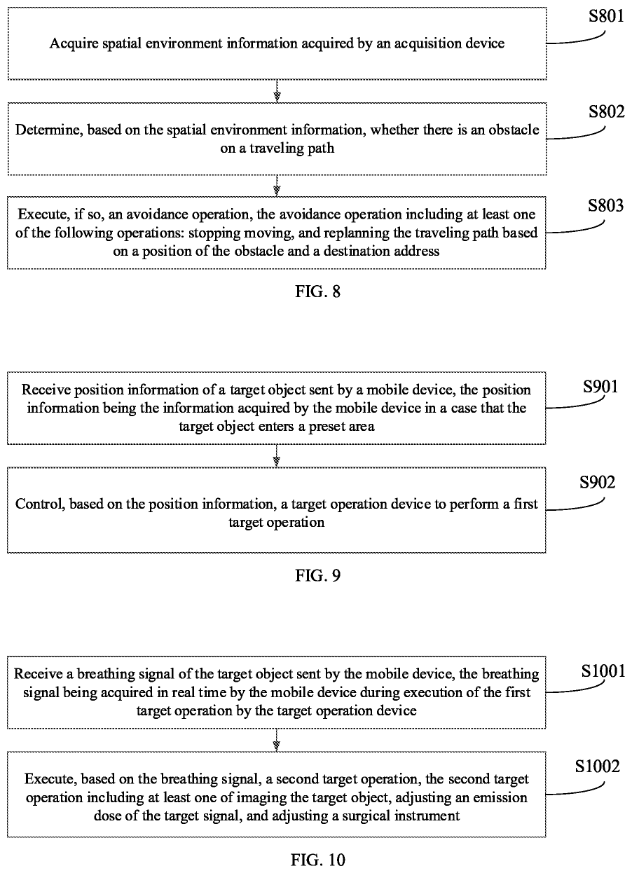

During the movement of the mobile device to a destination address, the mobile device may also perform an obstacle avoidance operation based on an image of a spatial environment where the mobile device is located. In an embodiment, as shown in FIG. 8, the above method further includes the following steps.

In step S801, spatial environment information acquired by an acquisition device is acquired.

Specifically, the mobile device acquires the spatial environment information acquired by the acquisition device. Optionally, the acquisition device may include a camera, a video camera, a laser or a lidar mounted on the mobile device, etc. Optionally, the mobile device may receive the spatial environment information acquired by the acquisition device in real time. Optionally, the above spatial environment information may include obstacle information on the traveling path of the mobile device and obstacle information on the wall of the traveling path of the mobile device. Exemplarily, if the above acquisition device is the camera, the above spatial environment information may be a space environment image. If the above acquisition device is the laser, the above spatial environment information may be a space echo signal, and the mobile device may determine the above spatial environment information based on the space echo signal, i.e., the laser emits a laser line, and the laser line is reflected back after encountering an obstacle, and then the mobile device may determine the spatial environment information based on the reflected laser line.

In step S802, whether there is an obstacle on a traveling path is determined based on the spatial environment information.

Specifically, the mobile device determines whether there is an obstacle on the traveling path based on the spatial environment information acquired above. Optionally, the obstacle on the traveling path of the mobile device may include a medical device, a technician, or a probe connected to the medical device. Optionally, if the above spatial environment information is the spatial environment image, the mobile device may input the acquired spatial environment image into a pre-trained recognition network, and use the recognition network to identify the environment image and determine whether there is an obstacle on the traveling path. Optionally, if the above spatial environment information is the spatial echo signal, the mobile device may determine that there is an obstacle on the traveling path after receiving the spatial echo signal. Optionally, the above traveling path may be a traveling path from the above operation room to the above operation area, or may be a traveling path from the above operation area to the above operation room.

In step S803, if so, an avoidance operation is executed. The avoidance operation includes at least one of the following operations: stopping moving, or replanning the traveling path based on a position of the obstacle and a destination address.

Specifically, if the mobile device determines that there is an obstacle on the traveling path, the avoidance operation is executed. The avoidance operation includes stopping moving, or replanning the traveling path based on the position of the obstacle and its own destination address. That is to say, when the mobile device determines that there is an obstacle on the traveling path, the mobile device will stop moving, or replan the traveling path based on the position of the obstacle and its own destination address to avoid the obstacle on the original traveling path.

In this embodiment, by acquiring the spatial environment image acquired by the acquisition device, the mobile device can promptly determine whether there is an obstacle on its own traveling path based on the spatial environment image. Further, in the case that it is determined that there is an obstacle on the traveling path, the mobile device will stop moving or replan the traveling path based on the position of the obstacle and the destination address to avoid the obstacle on the original traveling path and avoid a collision.

In a second aspect, in an embodiment, as shown in FIG. 9, a flow diagram illustrating an operation method for a medical device is provided. An example where the method is applied to the target host in FIG. 1 will be described. The method includes the following steps.

In step S901, position information of a target object sent by a mobile device is received. The position information is the information of the target object in a preset area acquired by the mobile device in a case that the target object enters the preset area.

The preset area may be a bed area, such as a scanning bed of a target operation device, or a treatment bed of the target operation device. The target operation device may include an X-ray digital radiography (DR) equipment, or a computed tomography (CT) equipment, or a magnetic resonance imaging (MRI) equipment, a positron emission tomography (PET) equipment, a medical electron linear accelerator, a gamma knife, a surgical robot, and so on. Specifically, the target host receives the position information of the target object sent by the mobile device. The position information of the target object is the information of the target object in the above preset area acquired by the mobile device in the case that the target object enters the above preset area. Optionally, the position information of the target object may be obtained by fusing local position information of the target object in the above preset area acquired by the mobile device from a plurality of angles. It should be noted that the transmission of the acquired position information of the target object in the preset area from the mobile device to the target host is real-time. Optionally, the mobile device may include a device having a moving capability such as an unmanned aerial vehicle, a mobile robot, a movable device, a wall slide rail robot, or the like, and this embodiment does not limit the specific device of the mobile device, as long as it has the moving capability. Optionally, a charging pile and a parking rack of the mobile device may be mounted at the door of an operation room.

In step S902, a target operation device is controlled to perform a first target operation based on the position information.

Specifically, the target host controls the target operation device to perform the first target operation based on the position information of the target object in the above preset area. Optionally, the first target operation in this embodiment may include emitting a target signal to an area of interest of the target object (for example, performing imaging or radiotherapy on the area of interest of the target object). For example, an X-ray signal may be emitted to the area of interest of the above target object, or radiation may be emitted to the area of interest of the above target object. Optionally, the first target operation in this embodiment may also include that the target operation device adjusts its own position. It should be noted that, if the target operation device is a radiotherapy equipment, and the application of radiotherapy includes a non-coplanar treatment, in this scenario, the treatment bed of the target operation device is at an unconventional angle. The mobile device may track the angle of the treatment bed and acquire a position and posture of the target object in real time at a current angle of the treatment bed, and send the acquired position and posture of the target object to the target host. Then the target host calculates a difference between the current position and posture of the target object and a planned position and posture, and compare the difference with a preset threshold. The threshold may be adjusted by a doctor, a physicist or a technician according to a condition of the target object and different imaging or treatment sites. If a real-time position deviation of the patient calculated by the target host exceeds the preset threshold, the target operation device will be controlled to terminate the treatment process, and the treatment process will be restarted after the technician redetermines the position accurately by moving the treatment bed (such as, a six-dimensional bed).

In this embodiment, since the target host controls the target operation device to perform the first target operation based on the position information of the target object in the preset area acquired by the mobile device, the target operation device can be accurately controlled based on the position information of the target object in the preset area, thereby ensuring the accuracy of the operation of the target operation device controlled by the target host.

In the scenario where the target host controls the target operation device to perform the first target operation based on the position information, on the basis of the above embodiment, in an embodiment, the above step S902 includes: controlling, based on the position information, the target operation device to emit a target signal to an area of interest of the target object, or to adjust its own position.

Specifically, the target host controls the target operation device to emit the target signal to the area of interest of the target object, or controls the target operation device to adjust its own position, based on the position information of the target object in the preset area. It should be noted that, the transmission of the acquired position information of the target object in the preset area from the mobile device to the target host is real-time. Optionally, the target host may compare the position information of the target object in the above preset area with the above preset reference position information to determine a matching result between the acquired position information and the above preset reference position information. The target host may also register the above acquired position information and the above preset reference position information to determine the matching result between the acquired position information and the above preset reference position information. Optionally, the matching result obtained by the target host may be that the above acquired position information matches the preset reference position information, or may be that the above acquired position information does not match the preset reference position information. Further, if the matching result obtained by the target host is that the above acquired position information matches the preset reference position information, the target host controls the above target operation device to emit the target signal to the area of interest of the target object. If the matching result obtained by the target host is that the above acquired position information does not match the preset reference position information, the target host controls the target operation device to adjust its own position. Optionally, the area of interest of the target object may be the abdomen of the target object, or the chest of the target object, etc. Optionally, the target signal emitted by the target operation device to the area of interest of the target object may include an X-ray signal, a pulse sequence, or radiation. It should be understood that the target signals emitted by different types of target operation devices are also different. If the target operation device is a DR equipment, the target signal emitted by the target operation device is the X-ray signal. If the target operation device is an MRI equipment, the target signal emitted by the target operation device is the pulse sequence. If the target operation device is the radiotherapy equipment, the target signal emitted by the target operation device is the radiation.

In this embodiment, the target host determines the matching result between the position information of the target object in the preset area and the preset reference position information, and controls the target operation device to emit the target signal to the area of interest of the target object, or to adjust its own position, based on the determined matching result. Since the target host controls the target operation device to perform the operation based on the determined matching result, the controlling the target operation device to perform the corresponding operation based on the position information of the target object in the preset area can ensure the accuracy of the operation performed by the target operation device controlled by the target host.

In some scenarios, the mobile device may also acquire a breathing signal of the target object in real time, and send the acquired breathing signal of the target object to the target host, so that the target host performs a corresponding operation based on the breathing signal of the target object during the above first target operation. In an embodiment, as shown in FIG. 10, a flow diagram illustrating another operation method is provided. Based on the above embodiment, as an optional implementation, the above method further includes the following steps.

In step S1001, a breathing signal of the target object sent by the mobile device is received. The breathing signal is acquired in real time during execution of the first target operation by the target operation device.

Specifically, the target host receives the breathing signal sent by the mobile device. The breathing signal is the breathing signal of the target object acquired by the mobile device in real time during the execution of the above first target operation by the above target operation device. Optionally, the breathing signal of the target object acquired by the mobile device in real time may be obtained by a depth image of the target object and an infrared image of the target object acquired by a depth camera and an infrared camera of the mobile device.

In step S1002, a second target operation is executed based on the breathing signal. The second target operation includes at least one of imaging the target object, adjusting an emission dose of the target signal, or adjusting a surgical instrument.

Specifically, the target host executes the second target operation based on the breathing signal of the above target object. The second target operation executed by the target host includes at least one of imaging the target object, adjusting the emission dose of the target signal, or adjusting the surgical instrument. It should be understood that, if the second target operation performed by the target host based on the breathing signal of the target object is to image the target object, the corresponding first target operation may include performing at least one of an X-ray scan, a CT scan, an MR scan, a PET scan, or an ultrasound scan. Optionally, in this embodiment, if the second target operation performed by the target host is to image the target object, the imaging method may be a prospective imaging method or a retrospective imaging method. For example, in CT imaging, the imaging scan may be performed at a specific respiratory phase based on the breathing signal, or the imaging scan data may be acquired at a plurality of respiratory cycles, and then a corresponding image may be reconstructed by extracting the imaging scan data at different respiratory phases. Optionally, in this embodiment, the target host controls the emission of the target signal based on the breathing signal of the target object. For example, in the radiotherapy, when the target object is monitored at the specific respiratory phase based on the breathing signal, the radiotherapy device is controlled to output a treatment beam. When the target object is not at the specific respiratory phase, the radiotherapy device is controlled to turn off the treatment beam. Optionally, in the radiotherapy, the target operation device performs the first and second target operations on the target object based on a four-dimensional (4D) radiotherapy plan. In this case, the target host may also monitor and control the output of the treatment beam of the radiotherapy device based on the breathing signal of the target object acquired in real time by the mobile device and the breathing signal in the four-dimensional radiotherapy plan. If a deviation between the real-time acquired signal and the signal in the four-dimensional radiotherapy plan exceeds a threshold, the radiotherapy device is controlled to stop the treatment.

In this embodiment, the target host receives the breathing signal of the target object acquired in real time by the mobile device during the execution of the first target operation by the target operation device, so that the target host can image the target object based on this breathing signal or adjust the emission dose of the target signal based on this breathing signal. As a result, the first target operation performed by the target operation device can be flexibly adjusted or the target object can be more accurately imaged based on the breathing signal of the target object.

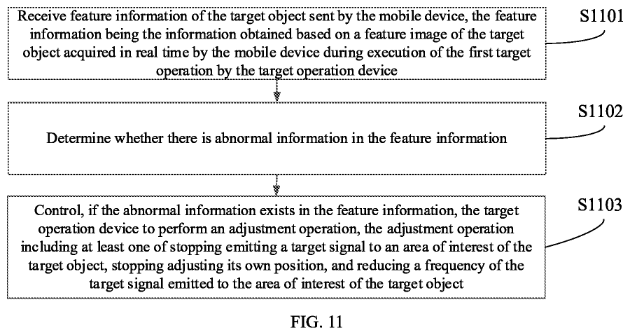

In some scenarios, the target host may also receive feature information of the target object sent by the mobile device, and control the target operation device to adjust the above first target operation based on the feature information of the target object. In an embodiment, as shown in FIG. 11, a flow diagram illustrating another operation method is provided. Based on the above embodiment, as an optional implementation, the above method further includes the following steps.

In step S1101, feature information of the target object sent by the mobile device is received. The feature information is the information obtained based on a feature image of the target object acquired in real time by the mobile device during the execution of the first target operation by the target operation device.

Specifically, the target host receives the feature information of the target object sent by the above mobile device. The feature information of the target object is the information obtained based on the feature image of the target object acquired in real time by the above mobile device during the execution of the first target operation by the target operation device. Optionally, the feature image of the target object acquired by the mobile device includes at least one of an RGB image of the target object, a depth image of the target object, or an infrared image of the target object. Optionally, the mobile device may acquire the RGB image of the target object through its own optical camera, acquire the depth image of the target object through its own depth camera, or acquire the infrared image of the target object through its own infrared camera. Optionally, the mobile device may perform feature extraction on the above feature image of the target object to obtain the feature information of the target object. For example, the mobile device may determine contour information of the target object through the depth image of the target object, and then determine the breathing signal of the target object. The mobile device may also determine a change in a temperature at the mouth and nose of the target object through the infrared image to obtain the breathing signal of the target object. Alternatively, the mobile device may determine the position information of the target object through the depth image of the target object, or the mobile device may determine facial expression information of the target object through the RGB image of the target object or the infrared image of the target object.

In step S1102, whether there is abnormal information in the feature information is determined.

Specifically, the target host determines whether there is the abnormal information in the received feature information of the target object. Optionally, the feature information of the target object includes the position information of the target object, facial expression of the target object, the breathing signal of the target object, etc. Exemplarily, the abnormal information in the feature information of the target object may include the position information of the target object deviating from preset position information, the facial expression of the target object being an abnormal facial expression, and the breathing signal of the target object being relatively rapid. Optionally, the target host may determine whether there is the abnormal information in the feature information of the target object through a recognition network, or determine whether there is the abnormal information in the feature information of the target object by classifying the feature information of the target object through a classifier.

In step S1103, the target operation device is controlled to perform an adjustment operation if the abnormal information exists in the feature information. The adjustment operation includes at least one of stopping emitting the target signal to the area of interest of the target object, stopping adjusting its own position, or reducing a frequency of the target signal emitted to the area of interest of the target object.

Specifically, if it is determined that the abnormal information exists in the feature information of the target object, the target host controls the above target operation device to perform the adjustment operation. The adjustment operation includes at least one of stopping emitting the target signal to the area of interest of the target object, stopping adjusting its own position, or reducing the frequency of the target signal emitted to the area of interest of the target object. Exemplarily, if the target host determines that the position information of the target object has deviated from the preset position information, the target host controls the target operation device to stop adjusting its own position and stop emitting the target signal to the area of interest of the target object. If the target host determines that the facial expression of the target object is the abnormal facial expression, or the breathing signal of the target object is relatively rapid, the target host may control the target operation device to stop emitting the target signal to the area of interest of the target object, or reduce the frequency of the target signal emitted to the area of interest of the target object.

In this embodiment, the target host receives the feature information of the target object sent by the mobile device, and determines whether there is the abnormal information in the feature information of the target object. In the case that the abnormal information exists in the feature information of the target object, the target host controls the target operation device to perform at least one of stopping emitting the target signal to the arca of interest of the target object, stopping adjusting its own position, or reducing the frequency of the target signal emitted to the area of interest of the target object. Since the target host controls the target operation device to perform the operation based on the feature information of the target object, the controlling the target operation device to perform the corresponding operation based on whether there is the abnormal information in the feature information of the target object can ensure the accuracy of the operation performed by the target operation device controlled by the target host.

The operation method provided by this disclosure will be described below using two specific scenarios of medical scanning and radiotherapy.

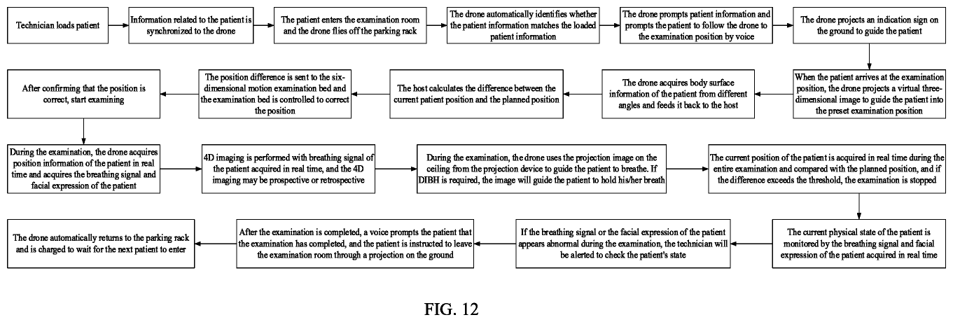

For the medical scanning scenario, as shown in FIG. 12, FIG. 12 is a flow diagram illustrating a medical scan using the operation method of the present disclosure. The operation process provided in FIG. 12 can be referred to the above embodiments of the method. The implementation principles and technical effects are similar, and will not be repeated here.

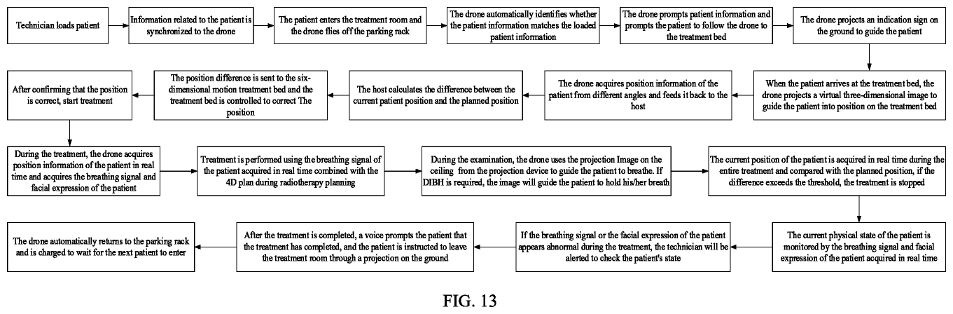

For the radiotherapy scenario, as shown in FIG. 13, FIG. 13 is a flow diagram illustrating a radiotherapy using the operation method of the present disclosure. The operation process provided in FIG. 13 can be referred to the above embodiments of the method. The implementation principles and technical effects are similar, which will not be repeated here.

It should be noted that FIGS. 12 and 13 are respectively described using the unmanned aerial vehicle the as the mobile device. The unmanned aerial vehicle is only an example, and the unmanned aerial vehicle in FIGS. 12 and 13 may also be replaced with other mobile devices. Optionally, the mobile device may also include at least one of a wall crawling robot, a ground walking robot, and a movable camera on a ceiling, which is not limited thereto.

It should be understood that although the individual steps in the flow diagrams of FIGS. 2 to 13 are shown sequentially as indicated by arrows, the steps are not necessarily performed sequentially in the order indicated by the arrows. Unless explicitly stated herein, the execution of these steps is not strictly limited in order and these steps can be performed in any other order. Moreover, at least some of the steps in the flow diagrams of FIGS. 2 to 13 may include a plurality of steps or a plurality of stages that are not necessarily performed at the same time, but may be performed at different times. The order in which these steps or stages are performed is not necessarily sequential, and these steps may be performed alternately or alternately with other steps or at least some of the steps or stages in other steps.

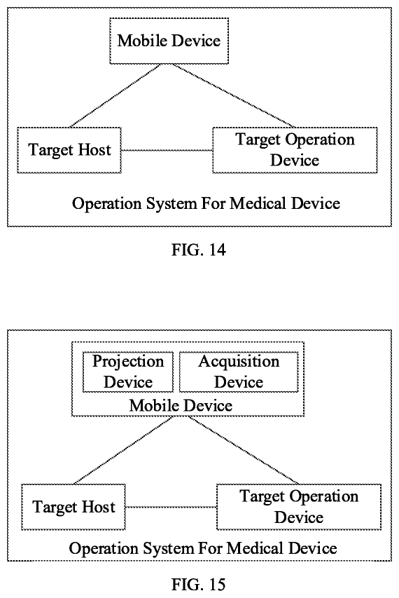

In a third aspect, in an embodiment, as shown in FIG. 14, an operation system for a medical device is provided. The operation system includes a target operation device, a mobile device, and a target host.

The target operation device is configured to perform a first target operation on an area of interest of a target object. The first target operation includes imaging and/or treatment. The mobile device is movable relative to the target operation device, and configured to present and/or acquire information related to the target object. The target host is configured to control the target operation device to perform the first target operation.

Specifically, the above operation system for the medical device includes the target operation device, the mobile device and the target host. The above target operation device is configured to perform the first target operation including the imaging and/or the treatment to the area of interest of the target object. Optionally, the first target operation in this embodiment may include emitting a target signal to the area of interest of the target object. For example, an X-ray signal may be emitted to the area of interest of the target object for CT or X-ray imaging, or a pulse sequence may be emitted to the area of interest of the target object for magnetic resonance imaging, or radiation may be emitted to the area of interest of the target object to perform radiotherapy on the target object. The radiation may include electrons, photons, protons or heavy ions. Alternatively, a surgical instrument may also be operated to operate on the area of interest of the above target object. Optionally, the first target operation in this embodiment may also include that the target operation device adjusts its own position, for example, adjustment of the position of a hospital bed. Optionally, the mobile device may control the target operation device to perform the above first target operation when position information of the target object on a preset area meets operation requirements. Exemplarily, taking the first target operation being to emit the target signal to the area of interest of the target object as an example, the mobile device may control the target operation device to emit the target signal to the area of interest of the target object in a case that the area of interest of the target object coincides with the emission position where the target operation device emits the target signal.

The above mobile device moves relative to the target operation device, and the mobile device is configured to present and/or acquire the information related to the target object. Optionally, the mobile device may present information that guides the target object to enter the preset area, or may also present information that guides the target object to leave an operation room. Optionally, the mobile device may also acquire position information of the target object or feature information of the target object during the process of the target operation device performing the first target operation on the target object, and then adjust the performed first target operation based on the acquired position information of the target object or the feature information of the target object. Optionally, the above mobile device includes at least one of a wall crawling robot, a ground walking robot, a movable camera on a ceiling, and an unmanned aerial vehicle, etc. Optionally, the above mobile device also includes a main body and a driving device that drives the main body to move.

The target host is configured to control the target operation device to perform the first target operation. Optionally, the target host may be integrated inside the mobile device, or may be a device that communicates with the mobile device and the target operation device, respectively. Optionally, the target host may control the target operation device to perform the first target operation based on the information related to the target object acquired by the mobile device.

In this embodiment, the operation system for the medical device includes the target operation device, the mobile device, and the target host. The target operation device is configured to perform the first target operation including the imaging and/or the treatment to the area of interest of the target object. The mobile device moves relative to the target operation device and is configured to present and/or acquire the information related to the target object. The target host is configured to control the target operation device to perform the first target operation. Since the mobile device moving relative to the target operation device can acquire three-dimensional position information of the target object on the target operation device, and the three-dimensional position information contains rich information, the target host can accurately control the target operation device to perform the first target operation in real time, thereby ensuring the accuracy of the first target operation performed by the target operation device.

In some scenarios, the mobile device needs to perform projection, so the mobile device may include a projection device. Based on the above embodiment, in an embodiment, as shown in FIG. 15, the above mobile device includes a projection device. The projection device is configured to project a first image for guiding the target object to position.

Specifically, the above mobile device includes the projection device configured to project the first image that guides the target object to position. Optionally, the first image may be an image that guides the target object to enter the preset area, or may also be an image that guides the target object how to position himself/herself on the hospital bed. Optionally, the first image may be a virtual two-dimensional or three-dimensional image.

Optionally, the projection device may be further configured to project a second image. The second image is configured to guide the target object to adjust his/her own breathing. For example, the target object is guided to perform a deep inspiration breath-hold technique (DIBH for short) by projecting the second image, and then perform the imaging or the treatment during the breath-hold period. It should be noted that, the mobile device guides the target object to adjust his/her own breathing through the second image, which may be the target object adjusting his/her own breathing frequency, or the target object adjusting his/her own breathing action. Optionally, the second image projected by the mobile device on the top of the preset position may be a breathing frequency signal, or a virtual three-dimensional breathing image. Optionally, when the mobile device guides the target object to adjust his/her own breathing action by projecting the second image on the top of the preset position, the mobile device may further guide the target object to adjust his/her own breathing action by broadcasting a corresponding guidance sound.

In this embodiment, the projection device included in the mobile device can project the first image that guides the target object to position. By projecting the first image, the target object can be guided to position accurately, thereby improving the accuracy of positioning the target object.

In some scenarios, the mobile device requires a corresponding acquisition device to acquire the position information of the target object. Based on the above embodiment, in an embodiment, referring to FIG. 15, the above mobile device further includes an acquisition device. The acquisition device is configured to acquire position information of the target object in a first orientation and a second orientation, respectively. The target host is configured to adjust a relative position between the target object and the target operation device, and/or control the target operation device to perform the first target operation, based on the position information of the first orientation and the position information of the second orientation.

Specifically, the above mobile device further includes the acquisition device. The acquisition device is configured to acquire the position information of the target object in the first orientation and the second orientation, respectively. The target host is configured to adjust the relative position between the target object and the target operation device, and/or control the target operation device to perform the first target operation based on the position information of the first orientation and the position information of the second orientation. It should be noted that the above first orientation and the second orientation are two different acquisition angles, respectively. For example, the first orientation and the second orientation may be opposite orientations or adjacent orientations. It should be understood that the first orientation and the second orientation are descriptions of the acquisition position of the acquisition device, and the acquisition device may acquire the position information of the target object at a plurality of positions, which is not limited to the first orientation and the second orientation. Optionally, the acquisition device may be further configured to acquire a breathing signal of the target object in real time, and send the breathing signal to the target host, so that the target host performs a second target operation based on the breathing signal. The second target operation includes at least one of imaging the target object, adjusting an emission dose of the target signal, or adjusting a surgical instrument. Optionally, the above acquisition device includes at least one of an RGB camera, a depth camera, an infrared camera, a voice device and a light emitting device. Optionally, the above mobile device also includes a trajectory generator. The trajectory generator is configured to generate a movement trajectory for the above mobile device. For example, the movement trajectory may be a position trajectory in which the mobile device moves from an initial position to the target operation device and runs through the operation process until the target object is guided to leave the operation room, or the movement trajectory may also be a position trajectory of a certain link in the entire workflow. For example, the movement trajectory may be a movement trajectory used to instruct the mobile device to move from the above first orientation to the above second orientation.

In this embodiment, the mobile device further includes the acquisition device, and the acquisition device can acquire the position information of the target object in the first orientation and the second orientation, respectively. Since the position information of the target object is acquired in the first orientation and the second orientation, respectively, the acquired position information of the target object contains rich information. Furthermore, the target host can accurately adjust the relative position between the target object and the target operation device, and/or control the target operation device to perform the first target operation based on the position information of the first orientation and the position information of the second orientation, thereby improving the accuracy of the target host adjusting the relative position between the target object and the target operation device and/or controlling the target operation device to perform the first target operation.

In some scenarios, there may be a plurality of mobile devices, which are arranged in a plurality of different positions, respectively. The mobile device may acquire position information of the target object at this plurality of different positions. Based on the above embodiment, in an embodiment, the above mobile device includes a first mobile device and a second mobile device. The first mobile device acquires the position information of the target object in the first orientation, and the second mobile device acquires the position information of the target object in the second orientation.

Specifically, the above mobile device includes the first mobile device and the second mobile device. The first mobile device acquires the position information of the target object in the first orientation, and the second mobile device acquires the position information of the target object in the second orientation. Optionally, in this embodiment, the first orientation and the second orientation may be different acquisition angles relative to the target object. It should be noted that the mobile device described in this embodiment including the first mobile device and the second mobile device is only an illustration of the number of the mobile device, and the above mobile device may include the plurality of mobile devices, which is not limited thereto.

In this embodiment, the mobile device includes the first mobile device and the second mobile device, and may acquire the position information of the target object from the plurality of different angles, so the acquired position information of the target object contains rich information. Furthermore, the target host can accurately adjust the relative position between the target object and the target operation device, and/or control the target operation device to perform the first target operation based on the position information of the first orientation and the position information of the second orientation, thereby improving the accuracy of the target host adjusting the relative position between the target object and the target operation device and/or controlling the target operation device to perform the first target operation.

In some scenarios, the mobile device may also guide the target object to move to the above preset operation area or guide the target object to leave the operation room. In an embodiment, the above mobile device is configured to provide a preset indication method to guide the target object to move to the preset operation area and/or leave the operation room.

Specifically, the above mobile device is configured to provide the preset indication method to guide the target object to move to the preset operation area and/or leave the operation room. Optionally, the preset indication method includes at least one of a voice prompt and an indication sign projected on the ground to guide the target object to move to the preset operation area and/or leave the operation room. It can be understood that an indication sign may also be arranged on the wall of the operation room to guide the target object to move to the above operation area, and the target object can be guided to move to the above operation area through this indication sign. An indication sign may also be arranged on the wall of the operation room to guide the target object to leave the operation room, and the target object can be guided to leave the operation room through this indication sign.

Further, as an optional implementation, before guiding the target object to move to the above operation area, the mobile device may verify identity information of the target object to ensure that the information of the entered target object is consistent with that of the object loaded by the technician. Specifically, the mobile device may be further configured to determine the identity information of the target object, and determine whether the identity information of the target object matches target identity information. If the identity information of the target object matches the target identity information, the target object is guided to move to the operation area through the above preset indication method. The target identity information is the identity information acquired from the target host. Optionally, the mobile device may also prompt the target object to verify their identity information by broadcasting the identity information of the target object through voice. Optionally, the mobile device may acquire the image of the wristband of the target object, and analyze the image of the wristband of the target object to obtain the identity information of the target object. Optionally, the mobile device may also instruct the target object to submit his/her identity information by sending a voice instruction to the target object.

In this embodiment, when detecting that the target object enters the operation room, the mobile device can quickly guide the target object to move to the operation arca through the preset indication method. In addition, the target object can be guided to move to the operation area more accurately through the indication method, thereby ensuring the accuracy of the target object moving to the operation arca.

During the above execution of the first target operation by the target operation device, the mobile device may also acquire position information of a moving part of the target object, and execute anti-collision monitoring on the moving part of the target object. In an embodiment, the mobile device is further configured to acquire position information of a moving part of the target object, and determine whether the target object collides with the target operation device based on the position information of the moving part. If so, the mobile device controls the target operation device to stop executing the first target operation.

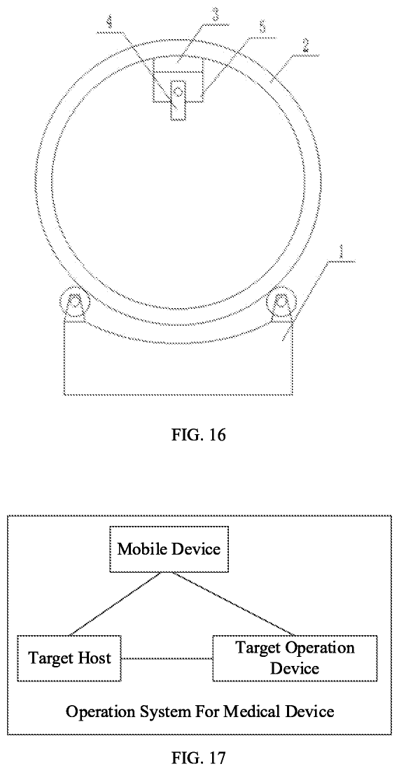

Specifically, during the execution of the above first target operation by the target operation device, the mobile device is further configured to acquire the position information of the moving part of the target object, and determine whether the target object collides with the target operation device based on the position information of the moving part of the target object. If the target object has collided with the target operation device, the target operation device is controlled to stop executing the first target operation. Optionally, the moving part of the target object may include the head of the target object, the legs of the target object, or the hands of the target object. Optionally, in the case of non-coplanar treatment, the hospital bed can be rotated to different positions around the vertical axis. Exemplarily, as shown in FIG. 16, FIG. 16 is a schematic diagram illustrating a non-coplanar treatment system. The non-coplanar treatment system includes a base 1, a rotating gantry 2, an arcuate guide rail 3, a treatment head module 4 and a treatment head stand 5. The rotating gantry 2 is mounted on the base 1 and can be rotated by a motor fixed on the base 1. The arcuate guide rail 3 with a rotation center of the rotating gantry 2 as a center is mounted on the inside of the rotating gantry 2 in an axial direction of the rotating gantry 2. The treatment head module 4 is mounted on the arcuate guide rail 3 through the treatment head stand 5, and can swing back and forth along the arcuate guide rail 3 with the rotation center of the rotating gantry 2 as the center. In this non-coplanar treatment system, since the treatment head module can swing back and forth along the arcuate guide rail with the rotation center of the rotating gantry as the center, the target object may collide with the target operation device, and the mobile device may acquire the position information of the target object in the process, and monitor whether the target object collides with the target operation device. In another embodiment, the treatment head module 4 may be mounted on the rotating gantry 2, and the hospital bed can rotate along a vertical axis in a horizontal plane, thereby achieving non-coplanar treatment. When the target object moves to different positions, the position information of the moving part of the target object may be acquired, and whether the target object collides with the target operation device may be monitored through the mobile device. Optionally, the mobile device may acquire an image of the moving part of the target object through the above acquisition device during the execution of the first target operation by the target operation device, and obtain the position information of the moving part of the target object based on the image of the moving part of the target object. Optionally, the mobile device may determine whether the target object collides with the target operation device based on a deviation between the position information of the moving part of the target object and the position information of the target operation device. Optionally, the mobile device may also determine whether the target object collides with the target operation device based on a distance between the position of the moving part of the target object and the position of the target operation device. Exemplarily, taking the target operation device being the medical electron linear accelerator and the moving part of the target object being the head of the target object as an example, the mobile device may determine whether the head of the target object collides with the medical electron linear accelerator based on the position information of the head of the target object. Optionally, the mobile device may send a control instruction to the target operation device to instruct the target operation device to stop executing the above first target operation.

In this embodiment, during the execution of the first target operation by the target operation device, the mobile device can determine whether the target object collides with the target operation device based on the position information of the moving part of the target object by acquiring the position information of the moving part of the target object, so that the target object can be timely performed anti-collision monitoring. In a case that it is determined that the target object has collided with the target operation device, the target operation device can be promptly controlled to stop executing the first target operation, thus avoiding risks to the target object and ensuring the safety of the target operation device performing the first target operation.

During the movement of the mobile device to a destination address, the mobile device may also perform an obstacle avoidance operation based on an image of a spatial environment where the mobile device is located. In an embodiment, the above mobile device is further configured to acquire spatial environment information acquired by the acquisition device, and determine whether there is an obstacle on the traveling path based on the spatial environment information. If so, the mobile device is configured to perform an avoidance operation. The avoidance operation includes at least one of the following operations: stopping moving, or replanning the traveling path based on a position of the obstacle and the destination address.

Specifically, the above mobile device is further configured to acquire the spatial environment information acquired by the acquisition device, and determine whether there is an obstacle on the traveling path based on the acquired spatial environment information. If the mobile device determines that there is an obstacle on the traveling path, the avoidance operation is performed. The avoidance operation includes at least one of the following operations: stopping movement, and replanning the traveling path based on the position of the obstacle and the destination address. That is to say, when the mobile device determines that there an obstacle on the traveling path, the mobile device will stop moving, or replan the traveling path based on the position of the obstacle and its own destination address to avoid the obstacle on the original driving path. Optionally, the acquisition device may include a camera, a video camera, a laser or a lidar mounted on the mobile device. Optionally, the mobile device may receive the spatial environment information acquired by the acquisition device in real time. Optionally, the above spatial environment information may include obstacle information on the traveling path of the mobile device and obstacle information on the wall of the traveling path of the mobile device. Exemplarily, if the above acquisition device is the camera, the above spatial environment information may be a space environment image. If the above acquisition device is the laser, the above spatial environment information may be a space echo signal, and the mobile device may determine the above spatial environment information based on the space echo signal, i.e., the laser emits a laser line, and the laser line is reflected back after encountering an obstacle, and then the mobile device may determine the spatial environment information based on the reflected laser line. Optionally, the obstacle on the traveling path of the mobile device may include a medical device, a technician, or a probe connected to the medical device. Optionally, if the above spatial environment information is the spatial environment image, the mobile device may input the acquired spatial environment image into a pre-trained recognition network, and use the recognition network to identify the environment image and determine whether there is an obstacle on the traveling path. Optionally, if the above spatial environment information is the spatial echo signal, the mobile device may determine that there is an obstacle on the traveling path after receiving the spatial echo signal. Optionally, the above traveling path may be a traveling path from the above operation room to the above operation area, or may be a traveling path from the above operation area to the above operation room.

In this embodiment, by acquiring the spatial environment image acquired by the acquisition device, the mobile device can promptly determine whether there is an obstacle on its own traveling path based on the spatial environment image. Further, in the case that it is determined that there is an obstacle on the traveling path, the mobile device will stop moving or replan the traveling path based on the position of the obstacle and the destination address to avoid the obstacle on the original traveling path and avoid a collision.

In a fourth aspect, in an embodiment, as shown in FIG. 17, an operation system for a medical device is provided. The operation system includes a mobile device, a target host, and a target operation device.

The mobile device is configured to perform the above-mentioned embodiments of the first aspect.

The target host is configured to perform the above-mentioned embodiments of the second aspect.

The target operation device is configured to perform a first target operation. The first target operation includes the target operation device emitting a target signal to an area of interest of the target object, and/or the target operation device adjusting its own position.

The operation system according to this embodiment may perform the above embodiments of the method. The implementation principles and technical effects are similar, which will not be repeated here.

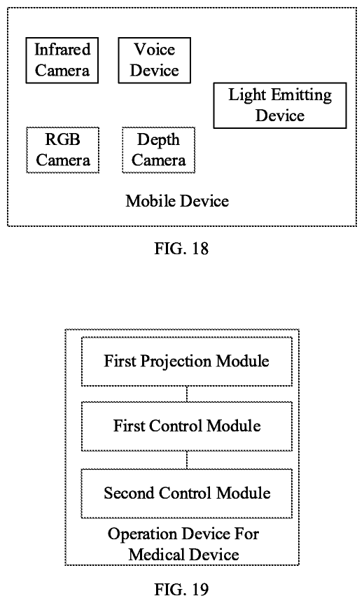

In a fourth aspect, in an embodiment, as shown in FIG. 18, a mobile device is provided. The mobile device is provided with at least one of an RGB camera, a depth camera, an infrared camera, a voice device, and a light emitting device.

It should be noted that FIG. 18 only illustrates the devices mounted on the mobile device, but not limits the devices mounted on the mobile device. Optionally, as an implementable implementation, the depth camera, the infrared camera, and the RGB camera used for facial recognition of the target object may be arranged on a same side to facilitate imaging the target object. The camera and the light emitting device may be arranged on the other side at a certain rotation angle, to ensure imaging and projection in different positions and directions. It should be noted that the above light emitting device is configured to emit structured light to the target object, and then the structured light is reflected back by the target object and received by the depth camera. Further, depth information of the target object can be calculated by an algorithm processing.

The mobile device according to this embodiment may perform the above embodiments of the method. The implementation principles and technical effects are similar, which will not be repeated here.

In an embodiment, as shown in FIG. 19, an operation device for a medical device is provided. The operation device includes a projection module, a first control module, and a second control module.

The projection module is configured to project, when it is detected that a target object reaches an operation area, a first image in the operation area. The first image is configured to guide the target object into a preset area.

The first control module is configured to acquire, in a case that the target object enters the preset area, position information of the target object in the preset area, and control, based on the position information, a target operation device to perform a first target operation.

The second control module is configured to acquire, during execution of the first target operation by the target operation device, feature information of the target object in real time, and control, based on the feature information, the target operation device to adjust the first target operation.

The operation device according to this embodiment may perform the above embodiment of the method. The implementation principles and technical effects are similar, which will not be repeated here.

Based on the above embodiment, optionally, the above first control module includes a first acquisition unit and a fusion unit.

The first acquisition unit is configured to acquire local position information of the target object in the preset area at each of at least one preset angle.

The fusion unit is configured to fuse the local position information acquired at each preset angle to obtain the position information.

The operation device according to this embodiment may perform the above embodiment of the method. The implementation principles and technical effects are similar, which will not be repeated here.

Based on the above embodiment, optionally, the above first control module includes a first determination unit and a first control unit.

The first determination unit is configured to determine a matching result between the position information and preset reference position information.

The first control unit is configured to control, based on the matching result, the target operation device to emit a target signal to an area of interest of the target object, or control the target operation device to adjust its own position.

The operation device according to this embodiment may perform the above embodiment of the method. The implementation principles and technical effects are similar, which will not be repeated here.

Based on the above embodiment, optionally, the above first control module includes a second control unit.

The second control unit is configured to send the position information to the target host, such that the target host controls, based on the position information and preset reference position information, the target operation device to emit a target signal to an area of interest of the target object, or to adjust its own position.

The operation device according to this embodiment may perform the above embodiment of the method. The implementation principles and technical effects are similar, which will not be repeated here.

Based on the above embodiment, optionally, the above second control module includes a second determination unit and a third control unit.

The second determination unit is configured to determine whether there is abnormal information in the feature information.

The third control unit is configured to control, if the abnormal information exists in the feature information, the target operation device to perform an adjustment operation. The adjustment operation includes at least one of stopping emitting a target signal to an area of interest of the target object, stopping adjusting its own position, or reducing a frequency of the target signal emitted to the area of interest of the target object.

The operation device according to this embodiment may perform the above embodiment of the method. The implementation principles and technical effects are similar, which will not be repeated here.

Based on the above embodiment, optionally, the above second control module includes a fourth control unit.

The fourth control unit is configured to send the feature information to a target host, such that the target host controls the target operation device to perform an adjustment operation based on the feature information. The adjustment operation includes at least one of stopping emitting a target signal to an area of interest of the target object, stopping adjusting its own position, or reducing a frequency of the target signal emitted to the area of interest of the target object.

The operation device according to this embodiment may perform the above embodiment of the method. The implementation principles and technical effects are similar, which will not be repeated here.

Based on the above embodiment, optionally, the above second control module includes a second acquisition unit and an obtainment unit.

The second acquisition unit is configured to acquire a feature image of the target object in real time. The feature image includes at least one of an RGB image of the target object, a depth image of the target object, or an infrared image of the target object.

The obtainment unit is configured to obtain, based on the feature image, the feature information.

The operation device according to this embodiment may perform the above embodiment of the method. The implementation principles and technical effects are similar, which will not be repeated here.

Based on the above embodiment, optionally, the above operation device further includes a guidance module.

The guidance module is configured to guide, during the execution of the first target operation by the target operation device, the target object to adjust its own breathing through a preset guidance method. The preset guidance method includes displaying a second image and/or broadcasting a voice.

The operation device according to this embodiment may perform the above embodiment of the method. The implementation principles and technical effects are similar, which will not be repeated here.

Based on the above embodiment, optionally, the above operation device further includes an acquisition module and a sending module.

The acquisition module is configured to acquire, during the execution of the first target operation by the target operation device, a breathing signal of the target object in real time.

The sending module is configured to send the breathing signal to the target host, such that the target host performs a second target operation based on the breathing signal. The second target operation includes at least one of imaging the target object, adjusting an emission dose of the target signal, or adjusting a surgical instrument.

The operation device according to this embodiment may perform the above embodiment of the method. The implementation principles and technical effects are similar, which will not be repeated here.

Based on the above embodiment, optionally, the above operation device further includes a guiding module.

The guiding module is configured to guide, when it is detected that the target object enters an operation room, the target object to move to the operation area through a preset indication method.

The operation device according to this embodiment may perform the above embodiment of the method. The implementation principles and technical effects are similar, which will not be repeated here.

Based on the above embodiment, optionally, the above guiding module includes a first guiding unit.

The first guiding unit is configured to broadcast a first voice prompt to guide the target object to move to the operation area.

The operation device according to this embodiment may perform the above embodiment of the method. The implementation principles and technical effects are similar, which will not be repeated here.

Based on the above embodiment, optionally, the above guiding module includes a second guiding unit.

The second guiding unit is configured to project a first indication sign to ground to guide the target object to move to the operation area.

The operation device according to this embodiment may perform the above embodiment of the method. The implementation principles and technical effects are similar, which will not be repeated here.

Based on the above embodiment, optionally, the above operation device further includes a first acquisition module and a determination module.

The first acquisition module is configured to acquire identity information of the target object.

The determination module is configured to determine whether the identity information matches target identity information. The target identity information is identity information acquired from the target host.

The guiding module is configured to guide, if the identity information matches the target identity information, the target object to move to the operation area through the preset indication method.

The operation device according to this embodiment may perform the above embodiment of the method. The implementation principles and technical effects are similar, which will not be repeated here.

Based on the above embodiment, optionally, the above operation device further includes a first voice broadcasting module.

The first voice broadcasting module is configured to broadcast the identity information of the target object through voice.

The operation device according to this embodiment may perform the above embodiment of the method. The implementation principles and technical effects are similar, which will not be repeated here.

Based on the above embodiment, optionally, the above operation device further includes a second voice broadcasting module.

The second voice broadcasting module is configured to broadcast, in a case that it is detected that the target operation device has completed the first target operation, a second voice prompt. The second voice prompt is configured to prompt the target object that the first target operation has been completed.

The operation device according to this embodiment may perform the above embodiment of the method. The implementation principles and technical effects are similar, which will not be repeated here.

Based on the above embodiment, optionally, the above operation device further includes a third projection module.

The third projection module is configured to project a second indication sign to the ground. The second indication sign is configured to guide the target object to leave the operation room.

The operation device according to this embodiment may perform the above embodiment of the method. The implementation principles and technical effects are similar, which will not be repeated here.

Based on the above embodiment, optionally, the above operation device further includes a second acquisition module, a first determination module, and a third control module.

The second acquisition module is configured to acquire, during the execution of the first target operation by the target operation device, position information of a moving part of the target object.

The first determination module is configured to determine, based on the position information of the moving part, whether the target object collides with the target operation device.

The third control module is configured to control, if it is determined that the target object collides with the target operation device, the target operation device to stop executing the first target operation.

The operation device according to this embodiment may perform the above embodiment of the method. The implementation principles and technical effects are similar, which will not be repeated here.

Based on the above embodiment, optionally, the above operation device further includes a third acquisition module, a second determination module, and an execution module.

The third acquisition module is configured to acquire spatial environment information acquired by an acquisition device.

The second determination module is configured to determine, based on the spatial environment information, whether there is an obstacle on a traveling path.

The execution module is configured to execute, if it is determined that there is the obstacle on the traveling path, an avoidance operation. The avoidance operation includes at least one of the following operations: stopping moving, or replanning the traveling path based on a position of the obstacle and a destination address.

The operation device according to this embodiment may perform the above embodiment of the method. The implementation principles and technical effects are similar, which will not be repeated here.

The specific limitations related to the operation device may be understood with reference to the limitations of the operation method above and will not be repeated here. The individual modules in the above operation device can be implemented in whole or in part by software, hardware and combinations thereof. Each of the above modules may be embedded in hardware form or independent of a processor in a computer device, or may be stored in software form on a memory in the computer device, so that the processor can be called to perform the operations corresponding to each of the above modules.

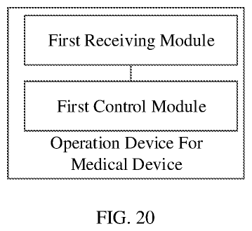

In an embodiment, as shown in FIG. 20, an operation device is provided. The operation device includes a first receiving module and a first control module.

The first receiving module is configured to receive position information of a target object sent by a mobile device. The position information is the information acquired by the mobile device in a case that the target object enters a preset area.

The first control module is configured to control, based on the position information, a target operation device to perform a first target operation.

The operation device according to this embodiment may perform the above embodiment of the method. The implementation principles and technical effects are similar, which will not be repeated here.

Based on the above embodiment, optionally, the above first control module includes a determination unit and a control unit.

The determination unit is configured to determine a matching result between the position information and preset reference position information.

The control unit is configured to control, based on the matching result, the target operation device to emit a target signal to an area of interest of the target object, or control the target operation device to adjust its own position.

The operation device according to this embodiment may perform the above embodiment of the method. The implementation principles and technical effects are similar, which will not be repeated here.

Based on the above embodiment, optionally, the above operation device further includes a second receiving module and an execution module.

The second receiving module is configured to receive a breathing signal of the target object sent by the mobile device. The breathing signal is acquired in real time by the mobile device during execution of the first target operation by the target operation device.

The execution module is configured to execute, based on the breathing signal, a second target operation. The second target operation includes at least one of imaging the target object, adjusting an emission dose of the target signal, or adjusting a surgical instrument.

The operation device according to this embodiment may perform the above embodiment of the method. The implementation principles and technical effects are similar, which will not be repeated here.

Based on the above embodiment, optionally, the above operation device further includes a third receiving module, a determination module, and a second control module.

The third receiving module is configured to receive feature information of the target object sent by the mobile device. The feature information is the information obtained based on a feature image of the target object acquired in real time by the mobile device during execution of the first target operation by the target operation device.

The determination module is configured to determine whether there is abnormal information in the feature information.

The second control module is configured to control, if the abnormal information exists in the feature information, the target operation device to perform an adjustment operation. The adjustment operation includes at least one of stopping emitting a target signal to an area of interest of the target object, stopping adjusting its own position, or reducing a frequency of the target signal emitted to the area of interest of the target object.

The operation device according to this embodiment may perform the above embodiment of the method. The implementation principles and technical effects are similar, which will not be repeated here.

The specific limitations related to the operation device may be understood with reference to the limitations of the operation method above and will not be repeated here. The individual modules in the above operation device can be implemented in whole or in part by software, hardware and combinations thereof. Each of the above modules may be embedded in hardware form or independent of a processor in a computer device, or may be stored in software form on a memory in the computer device, so that the processor can be called to perform the operations corresponding to each of the above modules.

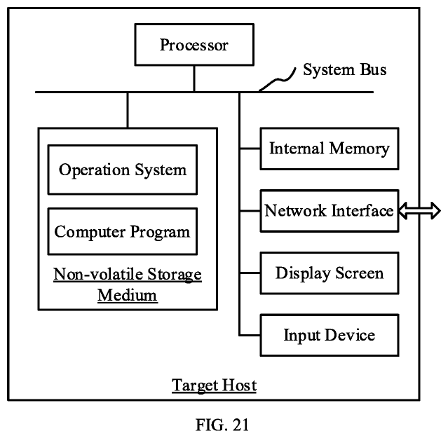

In an embodiment, as shown in FIG. 21, a target host is provided. The target host includes a processor and a memory storing a computer program. The computer program, when executed by the processor, causes the processor to perform the following steps.

Position information of a target object sent by a mobile device is received. The position information is the information acquired by the mobile device in a case that the target object enters a preset area.

A target operation device is controlled to perform a first target operation based on the position information.

In an embodiment, a computer-readable storage medium having a computer program stored thereon is provided. The computer program, when executed by a processor, causes the processor to perform the following steps.

A first image is projected in the operation area when it is detected that a target object reaches an operation area. The first image is configured to guide the target object into a preset area.

Position information of the target object in the preset area is acquired in a case that the target object enters the preset area, and a target operation device is controlled to perform a first target operation based on the position information.

Feature information of the target object is acquired in real time during execution of the first target operation by the target operation device, and the target operation device is controlled to adjust the first target operation based on the feature information.

In an embodiment, a computer-readable storage medium having a computer program stored thereon is provided. The computer program, when executed by a processor, causes the processor to perform the following steps.

Position information of a target object sent by a mobile device is received. The position information is the information acquired by the mobile device in a case that the target object enters a preset area.

A target operation device is controlled to perform a first target operation based on the position information.

A person of ordinary skill in the art can understand that implementation of all or part of the processes in the methods of the above embodiments can be completed by instructing the relevant hardware through a computer program. The computer program may be stored in a non-volatile computer-readable storage medium. When the computer program is executed, it may include the processes of the embodiments of the above methods. Any reference to memory, database or other medium used of the embodiments provided in the present disclosure may include at least one of a non-volatile and a volatile memory. The non-volatile memory may include a read-only memory (ROM), a magnetic tape, a floppy disk, a flash memory, or an optical memory, etc. The volatile memory may include a random-access memory (RAM) or an external cache memory, etc. As an illustration rather than a limitation, the random-access memory may be in various forms, such as a static random access memory (SRAM) or a dynamic random access memory (DRAM), etc.

The technical features in the above embodiments can be combined arbitrarily. For concise description, not all possible combinations of the technical features in the above embodiments are described. However, all the combinations of the technical features are to be considered as falling within the scope described in this specification provided that they do not conflict with each other.

The above-mentioned embodiments only describe several implementations of the present disclosure, and their description is specific and detailed, but should not be understood as a limitation on the patent scope of the present disclosure. It should be pointed out that for a person of ordinary skill in the art may further make variations and improvements without departing from the conception of the present disclosure, and these all fall within the protection scope of the present disclosure. Therefore, the protection scope of the present disclosure should be subject to the appended claims.

1. A method for a medical device, the method comprising:
projecting, in response to detecting that a target object reaches an operation area, a first image in the operation area, the first image being configured to guide the target object into a preset area;
acquiring, in response to detecting that the target object enters the preset area, position information of the target object in the preset area;
controlling, based on the acquired position information, a target operation device to perform a first target operation;
acquiring, during execution of the first target operation by the target operation device, feature information of the target object in real time;
controlling, based on the acquired feature information, the target operation device to adjust the first target operation.

2. The method of claim 1, wherein the acquiring the position information of the target object in the preset area comprises:
acquiring local position information of the target object in the preset area at each of at least one preset angle; and
fusing the local position information acquired at each of the at least one preset angle to obtain the position information.

3. The method of claim 1, wherein the controlling,
based on the acquired position information, the target operation device to perform the first target operation comprises:
controlling, based on the position information, the target operation device to emit a target signal to an area of interest of the target object, or to adjust a position of the target operation device.

4. (canceled)

5. The method of claim 1, wherein the controlling, based on the acquired feature information, the target operation device to adjust the first target operation comprises:
determining whether there is abnormal information in the feature information; and
controlling, if the abnormal information exists in the feature information, the target operation device to perform an adjustment operation, the adjustment operation comprising at least one of stopping emitting a target signal to an area of interest of the target object, stopping adjusting the position of the target operation device, or reducing a frequency of the target signal emitted to the area of interest of the target object.

6. (canceled)

7. The method of claim 1, wherein the acquiring the feature information of the target object in real time comprises:
acquiring a feature image of the target object in real time, the feature image comprising at least one of an RGB image of the target object, a depth image of the target object, or an infrared image of the target object; and
obtaining, based on the acquired feature image, the feature information.

8. The method of claim 1, further comprising:
guiding, during the execution of the first target operation by the target operation device, the target object to adjust breathing of the target object through a preset guidance method, the preset guidance method comprising displaying a second image or broadcasting a voice.

9. The method of claim 8, further comprising:
acquiring, during the execution of the first target operation by the target operation device, a breathing signal of the target object in real time; and
sending the breathing signal to the target host, such that the target host performs a second target operation based on the breathing signal, the second target operation comprising at least one of imaging the target object, adjusting an emission dose of the target signal, or adjusting a surgical instrument.

10. The method of claim 1, further comprising:
guiding, in response to detecting that the target object enters an operation room, the target object to move to the operation area through a preset indication method.

11. The method of claim 10, wherein the preset indication method comprises at least one of:
broadcasting a first voice prompt to guide the target object to move to the operation area; or
projecting a first indication sign to ground to guide the target object to move to the operation area.

12. (canceled)

13. The method of claim 11, wherein before the guiding the target object to move to the operation area through the preset indication method, the method further comprises:
acquiring identity information of the target object;
determining whether the identity information matches target identity information, wherein the target identity information is identity information acquired from the target host; and
guiding, if the identity information matches the target identity information, the target object to move to the operation area through the preset indication method.

14. (canceled)

15. The method of claim 1, further comprising:
broadcasting, in a case that it is detected that the target operation device has completed the first target operation, a second voice prompt, the second voice prompt being configured to prompt the target object that the first target operation has been completed.

16. The method of claim 15, further comprising:
projecting a second indication sign to the ground, the second indication sign being configured to guide the target object to leave the operation room.

17. The method of claim 1, further comprising:
acquiring, during the execution of the first target operation by the target operation device, position information of a moving part of the target object;
determining, based on the acquired position information of the moving part, whether the target object collides with the target operation device; and
controlling, in response to determining that the target object collides with the target operation device, the target operation device to stop executing the first target operation.

18. The method of claim 1, further comprising:
acquiring spatial environment information acquired by an acquisition device;
determining, based on the spatial environment information, whether there is an obstacle on a traveling path; and
executing, in response to determining that there is the obstacle on the traveling path, an avoidance operation, the avoidance operation comprising at least one of: stopping moving, or replanning the traveling path based on a position of the obstacle and a destination address.

19. A method for a medical device, the method comprising:
receiving position information of a target object sent by a mobile device, the position information being information acquired by the mobile device in a case that the target object enters a preset area; and
controlling, based on the received position information, a target operation device to perform a first target operation.

20-22. (canceled)

23. A system comprising:
a target operation device configured to perform a first target operation on an area of interest of a target object, the first target operation comprising imaging or treatment;
a mobile device movable relative to the target operation device, and configured to present and/or acquire information related to the target object; and
a target host configured to control the target operation device to perform the first target operation.

24. The system of claim 23, wherein the mobile device comprises a projection device configured to project at least one of a first image for guiding the target object to position, or a second image for guiding the target object to adjust the breathing of the target object.

25. The system of claim 23, wherein the mobile device further comprises an acquisition device configured to acquire position information of the target object in a first orientation and a second orientation, respectively; and
wherein the target host is configured to adjust a relative position between the target object and the target operation device, or control the target operation device to perform the first target operation, based on the position information acquired at the first orientation and the position information acquired at the second orientation.

26-27. (canceled)

28. The system of claim 25, wherein the acquisition device is further configured to acquire a feature image of the target object in real time, and the target host is configured to determine, based on the feature image, feature information of the target object, and control, based on the feature information, the target operation device to adjust the first target operation.

29. The system of claim 25, wherein the acquisition device is further configured to acquire a breathing signal of the target object in real time, and send the breathing signal to the target host, such that the target host performs a second target operation based on the breathing signal, the second target operation comprising at least one of imaging the target object, adjusting an emission dose of a target signal, and adjusting a surgical instrument.

30-42. (canceled)
